(19) United States
(12) Patent Application Publication
(10) Pub. No.: US 2010/0191096 A1
(43) Pub. Date: Jul. 29, 2010

(54) PARAMAGNETIC BIOMOLECULE COMPLEXES AND USES THEREOF IN THE ASSESSMENT OF ORGAN FUNCTION

(76) Inventor: Olav Tenstad, Bergen (NO)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/663,703

(22) PCT Filed: Jun. 6, 2008
(86) PCT No.: PCT/EP08/57093

§ 371 Date: Mar. 12, 2010

Related U.S. Application Data
(60) Provisional application No. 60/933,758, filed on Jun. 8, 2007.

(30) Foreign Application Priority Data
Jun. 8, 2007 (DK) PA 2007 00832

(51) Int. Cl. A61B 5/055; C07F 5/00; C07K 17/00; C12N 9/96; A61K 49/04; A61B 6/00

(52) U.S. Cl. 600/411; 534/15; 530/400; 530/367; 435/188; 530/316; 424/9.42; 600/431

(57) ABSTRACT

The present invention relates to complexes comprising one or more marker(s) and one or more biomolecules. The complexes according to the present invention have a variety of utilities, such as utility as a contrast agent in imaging methods, for example magnetic resonance imaging.

All patent and non-patent references cited in this application are hereby incorporated by reference in their entirety.

### FIELD OF INVENTION

The present invention relates to complexes comprising one or more marker(s) and one or more biomolecules. The complexes according to the present invention have a variety of utilities, such as utility as a contrast agent in imaging methods, for example magnetic resonance imaging.

This invention also relates to compositions, such as pharmaceutical compositions, comprising one or more complexes according to the invention, a kit-of-parts comprising the complexes, as well as methods for making and using the complexes and the compositions according to the invention.

### BACKGROUND

The main functions of the kidney are to regulate the amount of salt and water in the body to and remove waste products from plasma by excreting them into the urine. To accomplish this, 120 ml of plasma is normally filtered each minute in the glomeruli of the kidney.

A large number of kidney diseases, such as IgA nephritis, glomerulonephritis, chronic pyelonephritis and urinary retention, can lead to renal failure and subsequent decreased glomerular filtration rate (GFR). The decreased GFR may be associated with one or all of the following: proteinuria, inflammation, connective tissue formation and loss of functioning nephrons.

The decreased GFR leads in its turn to accumulation of waste products in the body, electrolyte and endocrine disturbances and hypertension. Hypertension will further damage the kidney and the outcome is a progressive deleterious process, which ultimately affects all organ systems and increases mortality.

If GFR falls below 60 ml/min, relative mortality is markedly increased, partly because decreased GFR is an independent risk factor for cardiovascular diseases [Manjunath et al (2003)ab]. It is estimated that as much as 5% of the world wide population or about 300 million people may be at risk, i.e. have a GFR lower than 60 ml/min. It follows that correct and timely measurements of kidney function in risk groups, e.g. patients with diabetes mellitus, hypertension, familial renal failure, nephrotoxic medication and proteinuria would represent a major socio-economic, medical and scientific gain.

The most commonly employed method to assess kidney function is a blood test for levels of creatinine. However, since creatinine levels in an individual depend not only kidney function, but also vary with age, sex, and race, creatinine levels are at best an approximation of the GFR in an individual.

Another method for measurement of GFR typically involves administering a substance which is freely filtered in the kidney to a subject and subsequently measuring the level of the substance in a recovered sample of body fluid (e.g. urine). Inulin has been used in this context as a marker of GFR. Measuring urinary clearance of inulin is laborious and time-consuming, and does not give information on individual kidney function.

One development along this principle is disclosed in U.S. Pat. No. 7,048,907 (Groman). In '907, a contrast agent is administered to a subject, and samples of body fluid (e.g. urine) are subsequently recovered from the subject. The release of this contrast agent to the body fluid is measured by neutron activation of the contrast agent in the recovered sample. This method shares the disadvantages of those cited for urinary clearance on inulin in being cumbersome, invasive and time-consuming.

Urography (visualising the urinary tract using a series of X-ray images) and scintigraphy (intravenous injection of radioactive substance and subsequent imaging of emitted gamma rays) are two further methods useful for visualizing kidney function. These methods both expose the subject to ionizing radiation. Furthermore, these methods do not allow calculation of regional glomerular filtration rate.

Magnetic resonance imaging (MR imaging) is a versatile imaging technology which has the advantages of being sensitive and using only non-ionizing radiation. Methods emplying MR imaging provide dynamic images of tissues and organs in a living body. In MR imaging, contrast agents are routinely administered to the subject in order to enhance images of organs or tissue.

One example of an organ in which MR imaging is used for functional imaging is the kidney. Functional MR imaging of the kidney relies at present on low molecular weight contrast agents. These agents are freely filtered in the kidneys and thus serve as markers of glomerular filtration rate. Examples of such markers are inulin and Gadolinium diethyllenetriamine pentaacetic acid (Gd-DPTA). These contrast agents have a short half-life, and provide no information on the source of proteinuria [Choyke et al (2006)].

Macromolecular contrast agents for the evaluation of specific renal parenchymal diseases in functional MR imaging of the kidney are known in the art. Macromolecular contrast agents were originally developed in order to increase the half-life of the contrast agents used, as this was desirable when performing high-resolution angiography [Choyke et al (2006)].

Ultrasmall particles of iron oxide (USPIO) are not filtered in the kidney, and such particles are therefore not normally observed when assessing kidney function. However, since monocytes and macrophages take up USPIO, and these subsequently localise at sites of inflammation, such sites of inflammation in the kidney may be visualised. This method would provide information as to where the kidney is damaged, but only if this damage to the kidney was associated with inflammation. However, no information would be available as to the reduction in GFR or the sources of proteinuria not involving inflammation [Choyke et al (2006)].

Dendrimers are organic molecules that are polymerised to form nanoparticles of precise sizes. Different sized dendrimers have different properties, in particular in reference to filtration/retention in the kidney. Kobayashi et al. 2005 discloses the use of Gd-labelled dendrimer nanoparticles for the injection into haematological malignancies to perform dynamic micro-magnetic resonance lymphangiography (micro-MRL).

Gd-MS-325 is a Gadolinium chelate which is injected intravenously. Once injected it binds to circulating albumin. After some time, the Gadolinium salt dissociates from the albumin and is filtered out with the urine [Choyke et al (2006)].

The use of iodinated Aprotinin for measurement of uptake in rat renal cortex is known [See references Tenstad et al, 1994ab, 1996; Wang et al 1995, 1996, 1997ab; Treeck et al 1997, 2002; Roald et al 2000, 2004ab, Baran et al 2003ab, 2006]. However, the use of MRI methods have not been disclosed.

### SUMMARY OF INVENTION

The present invention relates to new contrast agents in the form of complexes comprising one or more biomolecules linked to one or more markers.

The complexes according to the invention are preferably differentially accumulated in a target compartment. The contrast agents are thus useful in imaging techniques for visualising target compartments in an individual.

The methods of making the complexes make it possible to tailor complexes for use in visualising specific target compartments.

Examples of visualisation techniques comprise magnetic resonance imaging and radiograph-based imaging methods such as computer tomography-based imaging methods.

Examples where the complexes of the inventions are useful include use for functional assessment of the kidney, use for functional assessment of the small intestine, and use for assessment of the vasculature.

In the example of the kidney, the accumulation in the kidney enables sensitive and high resolution scans of the kidney to be performed. This in turn renders possible quantitative assessment of the glomerular filtration rate. Moreover, since a proteinuric kidney displays characteristic MRI scans depending on the source of proteinuria, the methods of the invention also makes it possible to gather detailed information on the underlying cause of decreased GFR. Furthermore, information from single kidneys can be gained.

### BRIEF DESCRIPTION OF THE DRAWINGS

The MRI hardware used to obtain the date in the figures described below is a Pharmascan 7T small animal MR scanner (from Bruker Biospin MRI GmbH, Ettlingen, Germany). The software is Paravision version 4 (from Bruker Biospin MR GmbH). Dynamic imaging was performed using a Flash sequence, with 2 mm slice thickness, 256×256 matrix, field of view (FOV) 6 cm, TR15.576 ms, TE3.7 ms, 250 evolution cycles, scan time 12 mins 27 sec. T1 weighted imaging was performed using a Flash T1 sequence, with 2 mm slice thickness, 256×256 matrix, FOV 6 cm, TR15.57 ms, TE3.7 ms, 10 averages, scan time 30 secs. T2 weighted imaging was performed using a Turbo RARE T2 sequence, with 2 mm slice thickness, 256×256 matrix, FOV 6 cm, TR4200 ms, TE36 ms, 3 averages, scan time 6 mins 47 secs.

FIG. 1 illustrates that Gd-DTPA-aprotinin yields several subpopulations of probes with decreasing pI corresponding to the amount of incorporated Gd-DTPA. The peak to the right shows a fraction that is left un-incorporated (co-elutes with native aprotinin) while the peak to the left represent the fraction which is most heavily incorporated with Gd-DTPA. Anionic Gd-DTPA-aprotinin is partially reabsorbed by the proximal tubular cells and is partially excreted into the urine. Cationic Gd-DTPA-aprotinin is filtered in the glomeruli and quantitatively accumulated in the proximal tubular cells in the same manner as native aprotinin. Gd-DTPA-aprotinin complexes can therefore be used as a MR imaging contrast agent for evaluation of single kidney function/glomerular filtration rate.

FIG. 2 illustrates that aprotinin, a 6.5 kDa polypeptide, is freely filtered in the glomeruli and quantitatively taken up into proximal tubular cells, close to its parent glomerulus by adsorptive endocytosis. Filtered aprotinin is then digested in the lysosomes and breakdown products can be detected in plasma 20 minutes after i.v. injection of labelled aprotinin (aprotinin*). By recording the uptake of aprotinin* in different layers of the renal cortex, detailed and accurate information of single kidney function can be obtained.

FIG. 3. Left panel: Plasma concentration relative to initial activity±1 SEM after i.v. injection of ¹²⁵I-aprotinin and ¹⁵³Gd-aprotinin.

FIG. 4. 153Gd-aprotinin was injected i.v into an anesthetised rat with a clamp on the right renal artery. The rat was killed 20 minutes thereafter and MRI of the excised kidneys was performed. Left: T2-weighted recording sequence providing anatomical details independent of function. Middle: T1-weighted recordings of the kidney with normal GFR showing a marked contrast enhancement in cortex compared to the recordings with the same protocol in the clamped kidney to the right. Table: Relative accumulation of Gd-aprotinin in various organs 20 minutes after i.v. injection. n=3.

FIG. 5. Size exclusion chromatogram of aprotinin (reference molecule) and Gd-aprotinin using a SW3000XL HPLC column from TosoHaas. The elution volume of molecular weight standards (IgM, BSA, ovalbumin, chymotrypsinogen A and aprotinin indicated by arrows.

FIG. 6. Size exclusion high performance chromatography (Toso Haas super SW3000, 4.6 mm diam×60 cm height eluted with 0.1 mol/L phosphate buffer+0.1 mol/L Na₂SO₄, pH 6.4, at 0.2 ml/min) of 1. Native Lysozyme (Ly), 2.Gd-DTPA-Lysozyme using standard protocol (Gd-Ly-1), 3. Gd-DTPA-Lysozyme using ¼ amount DTPA (Gd-Ly-2) and 4. Native ovalbumin (ovalb).

FIG. 7 illustrates T1 weighted MRI recordings of a rat kidney ex vivo (coronal section) 20 minutes after a five minute i.v. infusion of 1 ml Ringers solution with (A) and without (B) addition of 0.2 mg Gd-Ly. Panel C shows transverse sections of test-tubes with Ringer solution containing no Gd-Ly (big 12 ml tube in centre and small 0.8 ml tube to the left), 0.02 mg Gd-Ly (two small tubes in the middle) and 0.04 mg Gd-Ly (small tube to the right). The total amount Gd-Ly accumulated in the kidney displayed in panel A was about 10% of the injected dose (˜0.02 mg).

FIG. 8 illustrates size exclusion high performance chromatography (Toso Haas super SW3000, 4.6 mm diam×60 cm height eluted with 0.1 mol/L phosphate buffer+0.1 mol/L Na2SO4, pH 6.4, at 0.2 ml/min) of 1. Gd-DTPA-Chymotrypsinogen A (Gd-Chym) forming discrete complexes (a-d) with molecular weight ranging from 30-150 kDa and a continuum of larger complexes ranging from about 200 kDa to more than 900 kDa., 2. Native Chymotrypsinogen A (Chym) and 3. Plasma. Molecular weight of plasma albumin, plasma IgG, plasma IgM and native chymotrypsinogen A indicated.

FIG. 9

A: Plasma concentration relative to initial activity ±1 SEM after i.v. injection of ¹²⁵I-aprotinin and cationic ¹⁵³Gd-DTPA-aprotinin in 3 anaesthetized rats.

B: Relative accumulation of cationic ¹⁵³Gd-DTPA-aprotinin in various organs 20 minutes after i.v. injection in an anaesthetized rat.

FIG. 10

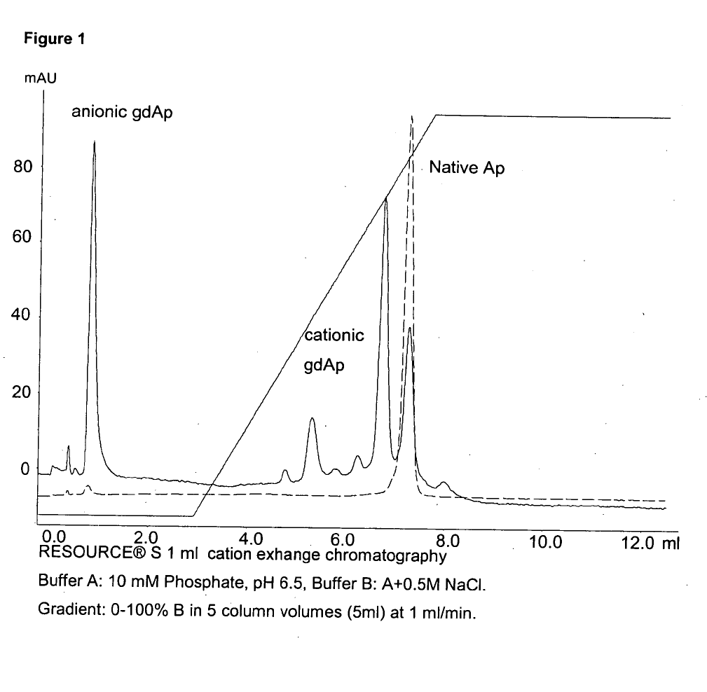

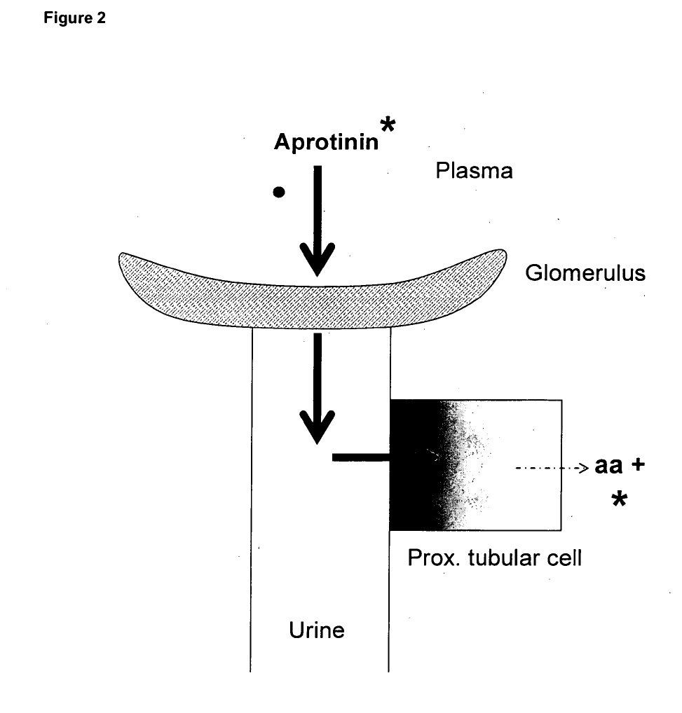

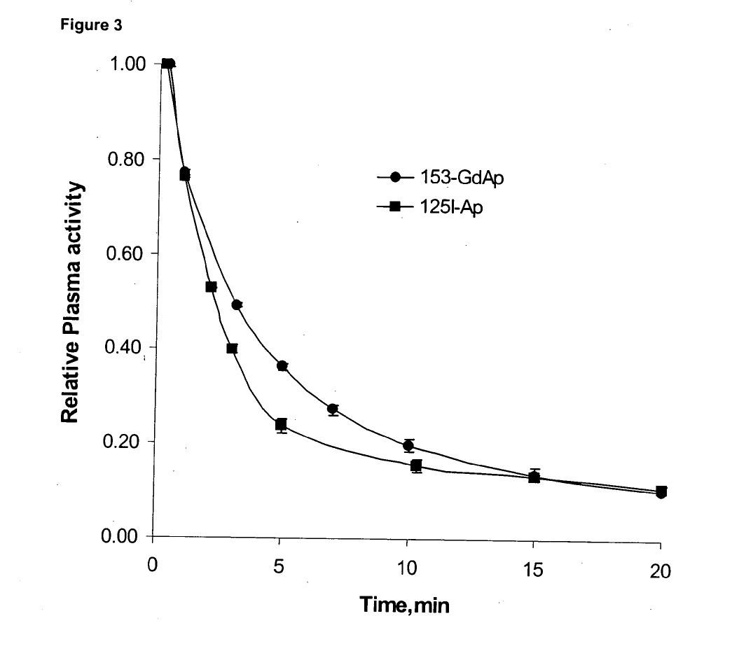

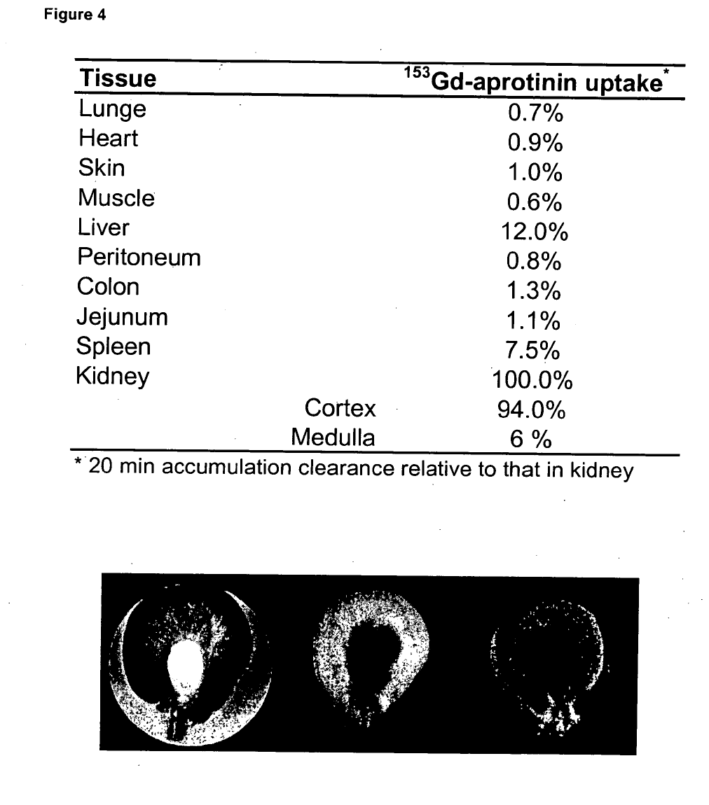

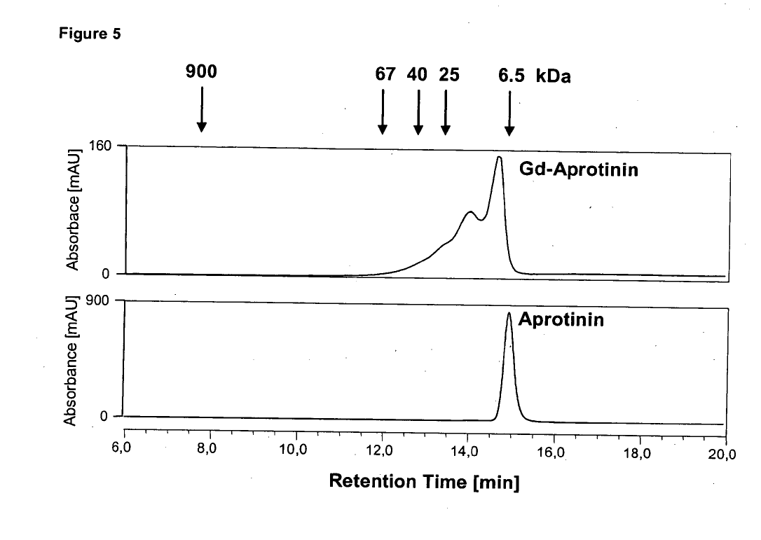

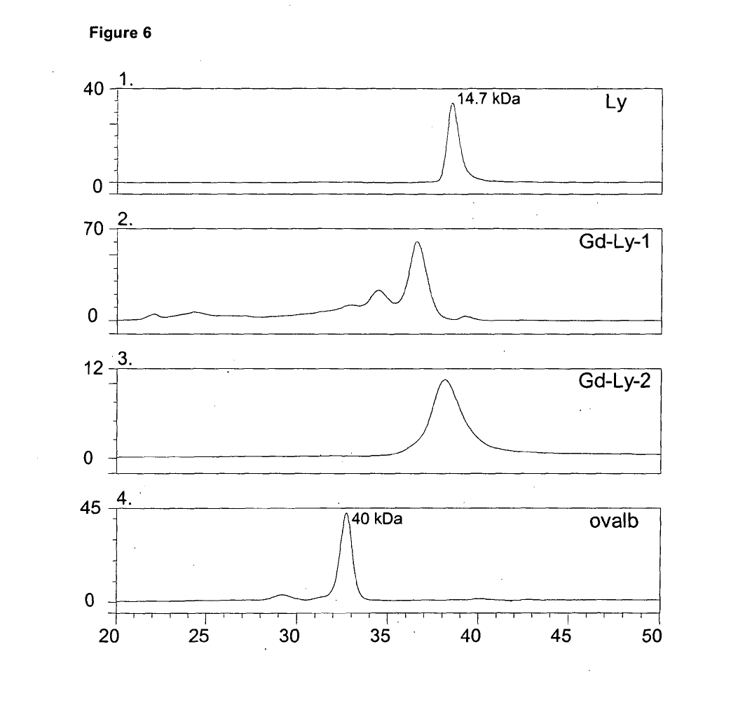

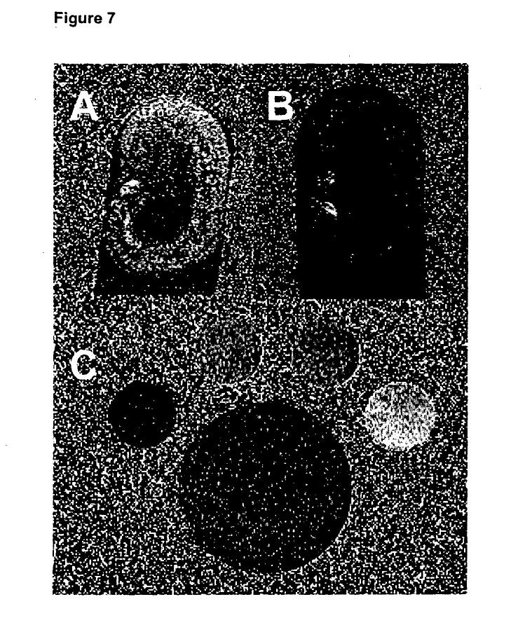

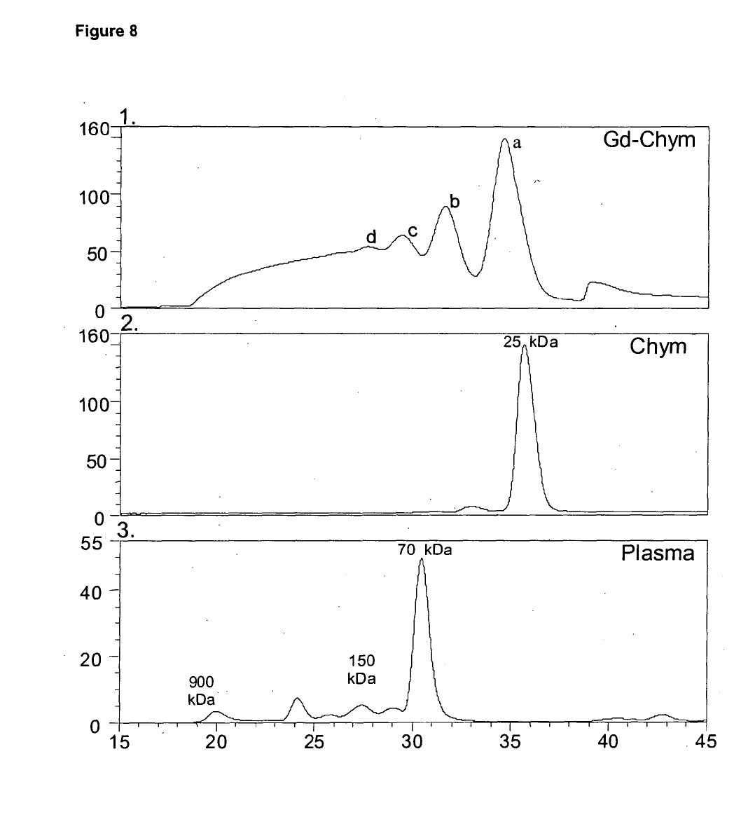

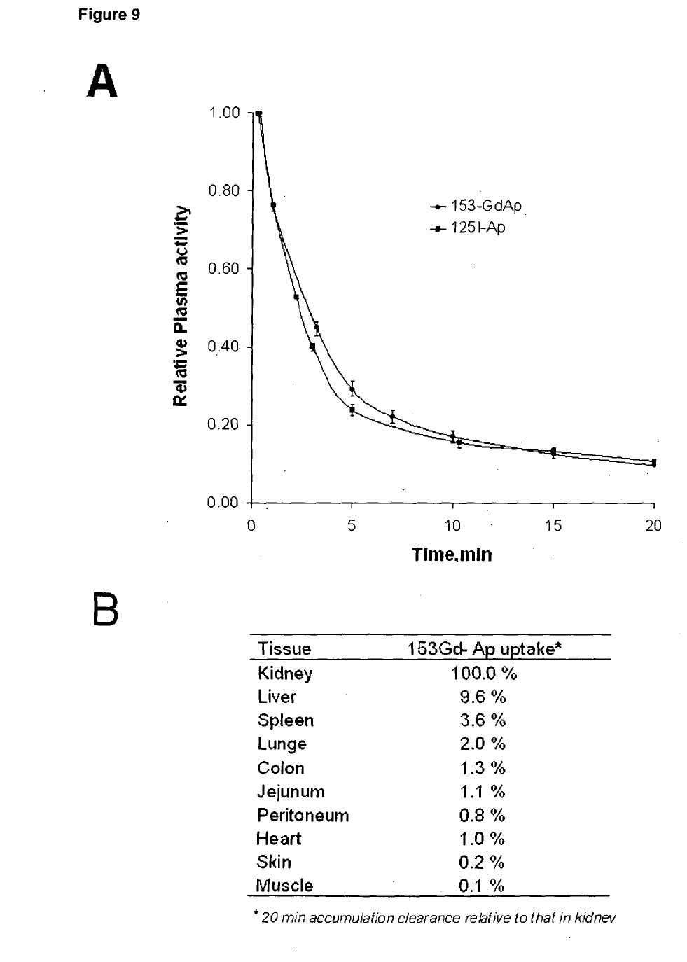

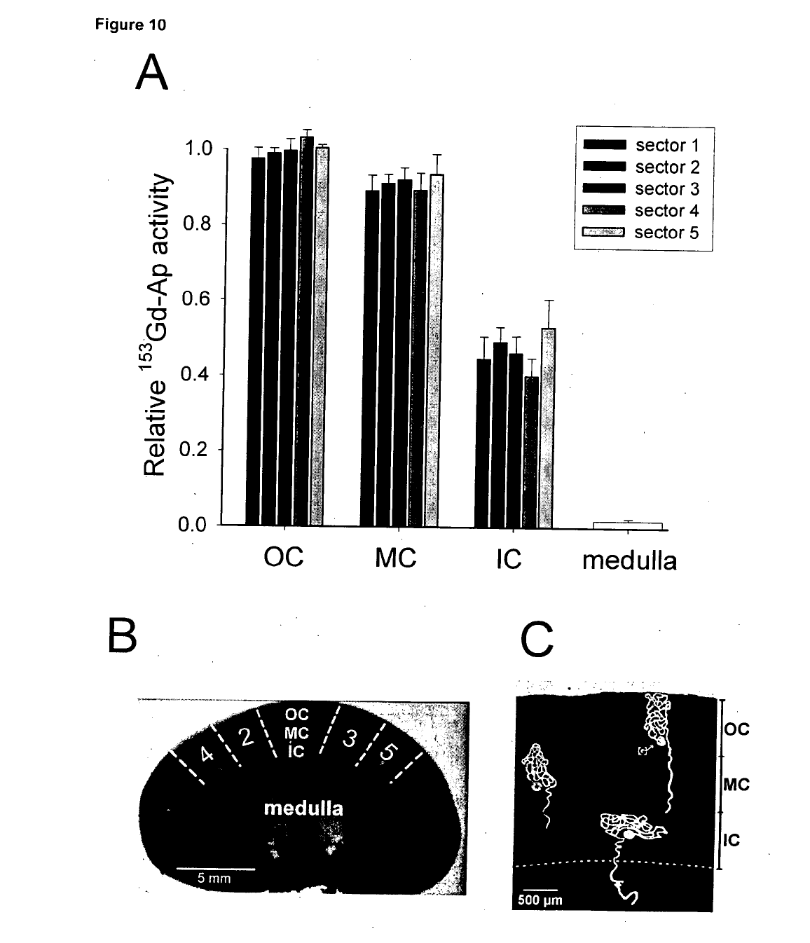

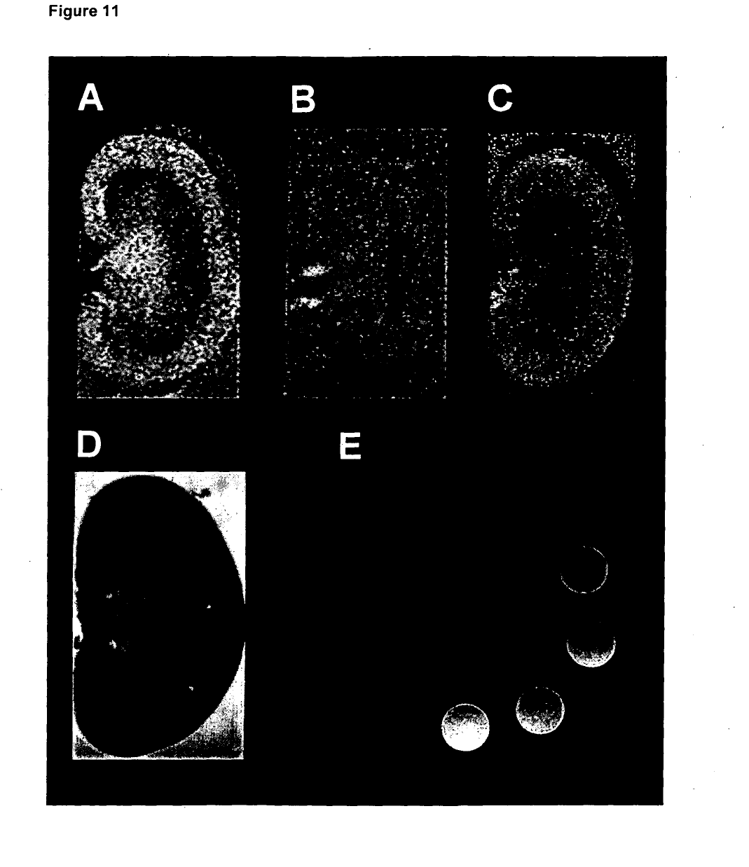

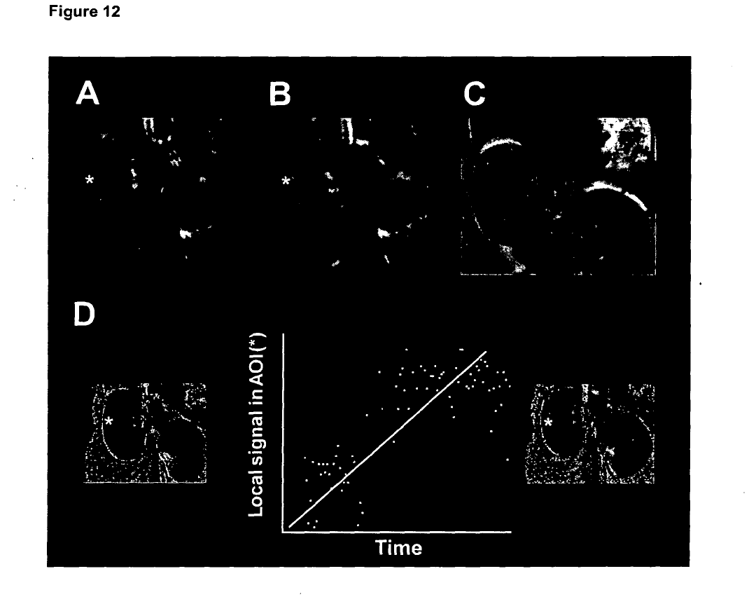

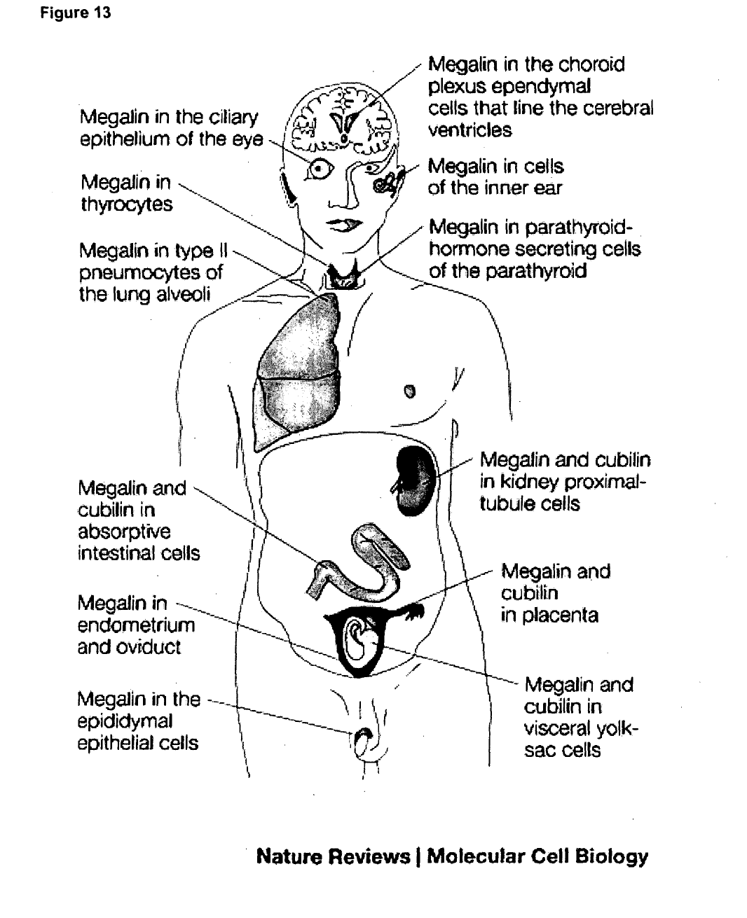

A: 20 minutes accumulation clearance relative to that in outer cortex (OC) of cationic ¹⁵³-Gd-DTPA-aprotinin in 4 formalin fixed kidneys. Tissue samples were obtained from five cortical sectors and from inner medulla as shown in panel B. Outer cortex (OC); Middle cortex (MC); Inner cortex (IC). Error bars; SEM.

B: Coronar T2-weighted magnetic resonance image of a rat kidney showing anatomical details. Overlay with cortical sectors and tissue sampling locations.

C: Microfil injected glomeruli and proximal convoluted tubules in formalin fixed kidneys. Proteins including Gd-DTPA-aprotinin are filtered in the glomeruli and taken up from the proximal tubular lumen by endocytosis and therefore accumulate only in the renal cortex. The lower activity of 153Gd-DTPA-aprotinin per gram inner cortex than in the two outer zones is mainly caused by inclusion of some tissue from outer medulla not containing proximal tubules. The black lumens represent glomeruli without microfil filling. Slightly modified from Baran et al 2003a.

FIG. 11

T1 weighted MRI recordings of coronal sections rat kidneys (ex vivo) 20 minutes after 30 seconds in vivo i.v. infusion of 800 μl Ringer solution containing ˜1 mg of Gd-DTPA-aprotinin (A), no contrast (B) and ˜1 mg Gd-DTPA-Lysozyme (C). Both complexes selectively enhanced the contrast in the renal cortex reflecting local glomerular filtration.

Panel D: T2 weighted MRI scan of the same area as in (B) providing anatomical details independent of function. Panel E: Transverse sections of 9 peripheral small 0.8 ml test-tubes containing clockwise from ten o′ clock: 1; Similar solution as injected in B (no contrast), 2-7; 10 μl, 50 μl, 100 μl, 200 μl, 300 μl and 400 μl of a similar solution as injected in (C). 8; Plasma from B (no contrast), 9: Plasma from C collected 20 minutes after Gd-DTPA-Ly infusion. The big 12 ml tube in center was filled with Ringer.

FIG. 12

In vivo T1 weighted coronal MRI scans in the same anaesthetized rat before (A) and 20 minutes after a 30 seconds i.v. infusion of 1 mg Gd-DTPA-Ly in 0.8 ml Ringer (B).

Panel C: Postmortal scan (improved image quality du to no respiratory movements) of the same rat as in (A) and (B).

Panel D: The increase in local signal intensity of an area of interest (AOI) as indicated by * was calculated by using flash puls-sequences with flip angles of 5, 10, 20 and 30 degrees to estimate R1 on voxel basis (inverse relaxation time, 1/T1). Examples of transformed images containing quantitative information are shown to the left (initial condition) and to the right (20 min after contrast enhancement) of the graph. Local glomerular filtration in different cortical regions may in principle be derived by calculating the time integrated local signal intensity divided by the time integrated signal intensity in systemic plasma.

FIG. 13: Organs comprising Megalin and Cubilin (Christensen & Birn 2002)

### DEFINITIONS

Unless specifically indicated otherwise, all technical and scientific terms used herein have the same meaning as commonly understood by those of ordinary skill in the art to which this invention belongs. For purposes of the present invention, the following terms are defined.

DPTA is Diethylene triamine pentaacetic acid
USPIO is ultra-small particles of iron oxide

The term “aprotinin” as used herein refers to a single-chain polypeptide which can be obtained at least from lung tissue of bovine species. As used herein, the term relates to a polypeptide of from 50 to 70 amino acids, such as about 58 amino acids. It is an inhibitor of certain proteolytic enzymes.

The term “lysozyme” as used herein refers to an enzyme of from 10 to 20 kDa, such as about 14.4 kilodalton enzyme, belonging to class EC 3.2.1.17 and consisting of a single polypeptide chain of about 120 to 140 amino acids, such as about 129 amino acids. The enzyme has four disulphide bridges.

The term “chymotrypsinogen A” as used herein refers to a precursor of the digestive enzyme chymotrypsin, which precursor has a molecular weight of from 20,000 kDa to 30,000 kDa, such as about 25,000 kDa, and an isoelectric point of from 8.7 to 9.5, such as a pI of about 9.1.

The term “Ovalbumin” refers to the major constituent of egg white. The ovalbumin protein of chickens is made up of about 385 amino acids and has a relative molecular mass of about 45 kD. Ovalbumin from sources others than chickens are also envisaged.

The term “biomolecule” as used herein refers to a natural compound or a synthetic, biocompatible compound, such as a polypeptide or any other chemical compound that originally occurs in living organisms, irrespective of the means by which the biomolecule is produced, and including all variants of said chemical compound. For example, aprotinin, aprotinin fragments, a chimera of aprotinin and a second protein, and glycosylated aprotinin are all biomolecules within the meaning used herein.

The term “linker” as used herein refers to a residue or chemical bond separating at least two species, such as a biomolecule and a marker. The species may be retained at an essentially fixed distance, or the linker may be flexible and allow the species some freedom of movement in relation to each other. The link can be a covalent bond or a non-covalent bond. Linked species include e.g. a complementing element and a functional entity of a building block, neighbouring coding elements of a template, neighbouring complementing elements of a complementing template, and neighbouring functional groups of a templated molecule.

The term “complex” herein is used to refer to both the form B-X-M and (B-X-M)ⁿ, where n is a positive integer.

The term supracomplex is used to denote (B-X-M)ⁿ, where n is a positive integer.

The term “contrast agent” is used herein to refer to a compound or complex capable of improving the visibility of body structures in imaging techniques, such as Magnetic resonance imaging, or radiograph based methods such as Computed tomography—based methods. The term as used herein is synonymous with “labelling agent”. The complexes of the invention can have the formula B-X-M, or be linked together to form supracomplexes (B-X-M)ⁿ, where n=a positive integer. The term contrast agent comprises both forms. The term contrast agent herein is used interchangeably with the term complexes.

The term “macromolecular contrast agents” as used herein refers to compounds with a molecular weight of preferably more than 1 kDa and preferably less than 1500 kDa.

The term “engineered” is used herein to refer to molecules which are synthesised or are in any way modified in order to derivatise a natural molecule.

The term “magnetic resonance imaging” is used herein to refer to the use of magnetic resonance to capture images, particularly of a living body. It is to be understood that the term includes both dynamic and still images received as a result of the technique.

The term “computed tomography” is ised herein to refer to a medical imaging method employing tomography where digital geometry processing is used to generate a three-dimensional image of the internals of an object from a large series of two-dimensional X-ray images taken around a single axis of rotation. The term as used herein is non-exclusive, and includes CT-based methods and combination methods, such as PET/CT (positron emisson tomography).

The term “target compartment” as used herin may refer to any location in an individual. Examples of target compartments may be:
an organ e.g. the heart,
a tissue (e.g. the cortex of the kidney),
a compartment (e.g. the lumen of an artery)

The term “individual” as used herein refers to any body, living or dead, of any species.

The term “normal” as used herien refers to an individual assessed to be normal by general standards.

The term “differential accumulation” is used herein to describe the situation where there is a difference in the amount of marker in the target compartment relative to the surroundings. E.g., an accumulation of the complex in kidney cortex, or in the case of leaky vasculature, where in a normal situation the complex would be retained, the loss of complex is indicative of an abnormal situation.

The term “radioactive” is used herein to describe a substance which gives off, or is capable of giving off, radiant energy in the form of particles (alpha or beta radiation) or rays (gamma radiation) by the spontaneous disintegration of the nuclei of atoms. Radioisotopes of elements lose particles and energy through the process of radioactive decay. Elements may decay into different atoms or a different state of the same atom.

The term “Glomerular filtration rate” (GFR) is used herein to refer to the rate at which a plasma water including freely filterable solutes is filtered through the glomeruli of an individual.

The term “proteinuria” is used herein to refer to a clinical state where protein is found in the urine.

### DETAILED DESCRIPTION OF THE INVENTION

### Methods of Making Complexes

Complexes according to the present invention may be illustrated by the general formula B-X-M, wherein B is indicative of one or more biomolecules, X is an optional linker moiety, for example a chelator, and M is a marker moiety. M may for example be a paramagnetic moiety, a radioactive label or a fluorescent label.

The complexes of the invention are synthesised by a novel and inventive method which comprises the steps of:

Providing the biomolecule based on the desired target compartment
Providing the marker moiety
Linking the biomolecule to the marker moiety via a linker
Selecting the complex based on pI, size and specificity.

### The Biomolecule Moiety

The biomolecule moiety of the complex may be selected in order to achieve a differential accumulation of the complex in or near the target compartment. The differential accumulation may be an increased amount of complex present in or near the target compartment as compared to the normal case. Differential accumulation may also be a diminished amount of complex in or near the target organ relative to the normal case.

In other embodiments, the differential accumulation is a result of passive forces. Normally, large anionic complexes are retained in the lumen of vasculature. In the case of leaky vasculature, such complexes are leaked to the surrounding tissue. Thus, visualising the leakage of these complexes is indicative of an particular situation in that vasculature. E.g that the vessels are newly formed, or indicative of some pathology.

In some embodiments, the differential accumulation is achieved by selecting a biomolecule which specifically interacts with components of the target compartment, and then either retained or eliminated.

Further particular embodiments include biomolecules engineered so as to confer to the complex the ability to accumulate in the target organ, or to enhance an integral ability of the biomolecule to do so.

The one or more biomolecules of the invention are preferably selected from nucleic acids, polypeptides (hereunder glycosylated and otherwise post-translationally modified polypeptides), polysaccharides and/or lipids and/or mixtures thereof.

In one embodiment the biomolecule is a nucleic acid with a length of from 1 nucleic acid to 5,000 nucleic acids, such as from 1 nucleic acid to 5 nucleic acids, for example from 5 to 10 nucleic acids, such as from 10 nucleic acid to 15 nucleic acids, for example from 15 to 20 nucleic acids, such as from 20 nucleic acid to 25 nucleic acids, for example from 25 to 30 nucleic acids, such as from 30 nucleic acid to 35 nucleic acids, for example from 35 to 40 nucleic acids, such as from 40 nucleic acid to 45 nucleic acids, for example from 45 to 50 nucleic acids, such as from 50 nucleic acid to 75 nucleic acids, for example from 75 to 100 nucleic acids, such as from 100 nucleic acid to 150 nucleic acids, for example from 150 to 200 nucleic acids, such as from 200 nucleic acid to 250 nucleic acids, for example from 250 to 300 nucleic acids, such as from 300 nucleic acid to 350 nucleic acids, for example from 350 to 400 nucleic acids, such as from 400 nucleic acid to 450 nucleic acids, for example from 450 to 500 nucleic acids, such as from 500 nucleic acid to 600 nucleic acids, for example from 600 to 700 nucleic acids, such as from 700 nucleic acid to 800 nucleic acids, for example from 800 to 900 nucleic acids, such as from 900 nucleic acid to 1000 nucleic acids, for example from 1000 to 1500 nucleic acids, such as from 1500 nucleic acid to 2000 nucleic acids, for example from 2000 to 2500 nucleic acids, such as from 2500 nucleic acid to 3000 nucleic acids, for example from 3000 to 3500 nucleic acids, such as from 3500 nucleic acid to 4000 nucleic acids, for example from 4000 to 4500 nucleic acids, such as from 4500 to 5000 nucleic acids.

In one embodiment the biomolecule is a polypeptide with a length of from 1 amino acid to 2000 amino acids, such as from 1 to 5 amino acids, for example from 5 to 10 amino acids, such as from 10 to 15 amino acids, for example from 15 to 20 amino acids, such as from 20 to 25 amino acids, for example from 25 to 30 amino acids, such as from 30 to 35 amino acids, for example from 35 to 40 amino acids, such as from 40 to 45 amino acids, for example from 45 to 50 amino acids, such as from 50 to 75 amino acids, for example from 75 to 100 amino acids, such as from 100 to 150 amino acids, for example from 150 to 200 amino acids, such as from 200 to 250 amino acids, for example from 250 to 300 amino acids, such as from 300 to 400 amino acids, for example from 400 to 500 amino acids, such as from 500 to 600 amino acids, for example from 600 to 700 amino acids, such as from 700 to 800 amino acids, for example from 800 to 900 amino acids, such as from 900 to 1000 amino acids, for example from 1000 to 1200 amino acids, such as from 1200 to 1400 amino acids, for example from 1400 to 1600 amino acids, such as from 1600 to 1800 amino acids, for example from 1800 to 2000 amino acids.

In one embodiment the one or more polysaccharides comprises one or more branched polymers. The present invention relates to homopolysaccharides as well as heteropolysaccharides and mixtures thereof. The polysaccharides may comprise glucose and/or mannose and/or galactose and/or fructose and/or maltose and/or sucrose and/or lactose and/or cellulose.

Polysaccharides have a general formula of Cₙ(H₂O)ₙ₋₁ where n is usually a large number between 200 and 2500. Considering that the repeating units in the polymer backbone are often six-carbon monosaccharides, the general formula can also be represented as (C₆H₁₀O₅)ₙ where n={40 . . . 3000}.

In one embodiment the biomolecule is a polysaccharide with the formula of Cₙ(H₂O)ₙ₋₁ where n is number between 200 and 2500, such as from 200 to 300, for example from 300 to 400, such as from 400 to 500, for example from 500 to 600, such as from 600 to 700, for example from 700 to 800, such as from 800 to 900, for example from 900 to 1000, such as from 1000 to 1100, for example from 1100 to 1200, such as from 1200 to 1300, for example from 1300 to 1400, such as from 1400 to 1500, for example from 1500 to 1600, such as from 1600 to 1700, for example from 1700 to 1800, such as from 1800 to 1900, for example from 1900 to 2000, such as from 2000 to 2100, for example from 2100 to 2200, such as from 2200 to 2300, for example from 2300 to 2400, such as from 2400 to 2500.

In another embodiment the biomolecule is a polysaccharide with the formula (C₆H₁₀O₅)ₙ where n from 40 to 3000, such as from 40 to 100, for example from 100 to 200, such as from 200 to 300, for example from 300 to 400, such as from 400 to 500, for example from 500 to 600, such as from 600 to 700, for example from 700 to 800, such as from 800 to 1000, for example from 1000 to 1200, such as from 1200 to 1400, for example from 1400 to 1600, such as from 1600 to 1800, for example from 1800 to 2000, such as from 2000 to 2200, for example from 2200 to 2400, such as from 2400 to 2600, for example from 2600 to 2800, such as from 2800 to 3000.

In one embodiment the biomolecule is a lipid. The lipid can in one embodiment be selected from the group consisting of any fat-soluble (lipophilic), naturally-occurring molecule, such as fats, oils, sterols, fat-soluble vitamins (such as vitamins A, D, E and K), phospholipids, monoglycerides, diglycerides, triglycerides, fatty acid, fatty acids derivates, sterol-containing metabolites such as cholesterol.

The Lipids may be broadly defined as hydrophobic or amphiphilic small molecules that originate entirely or in part from two distinct types of biochemical subunits or “building blocks”: ketoacyl and isoprene groups. Using this approach, lipids may be divided into eight categories:fatty acyls, glycerolipids, glycerophospholipids, sphingolipids, saccharolipids and polyketides (derived from condensation of ketoacyl subunits); and sterol lipids and prenol lipids (derived from condensation of isoprene subunits). Fatty acyls (including fatty acids) are a diverse group of molecules synthesized by chain-elongation of an acetyl-CoA primer with malonyl-CoA or methylmalonyl-CoA groups. The carbon chain may be saturated or unsaturated, and may be attached to functional groups containing oxygen, halogens, nitrogen and sulfur. Examples of biologically interesting fatty acyls are the eicosanoids which are in turn derived from arachidonic acid which include prostaglandins, leukotrienes, and thromboxanes. Other major lipid classes in the fatty acyl category are the fatty esters and fatty amides. Fatty esters include important biochemical intermediates such as wax esters, fatty acyl thioester coenzyme A derivatives, fatty acyl thioester ACP derivatives and fatty acyl carnitines. The fatty amides include N-acyl ethanolamines such as anandamide.

Glycerolipids are composed mainly of mono-, di- and tri-substituted glycerols, the most well-known being the fatty acid esters of glycerol (triacylglycerols), also known as triglycerides. Additional subclasses are represented by glycosylglycerols, which are characterized by the presence of one or more sugar residues attached to glycerol via a glycosidic linkage. Examples of structures in this category are the digalactosyldiacylglycerols and seminolipid.

Glycerophospholipids are also referred to as phospholipids. Glycerophospholipids may be subdivided into distinct classes, based on the nature of the polar headgroup at the sn-3 position of the glycerol backbone in eukaryotes and eubacteria or the sn-1 position in the case of archaebacteria. Examples of glycerophospholipids found in biological membranes are phosphatidylcholine (also known as PC or GPCho, and lecithin), phosphatidylethanolamine (PE or GPEtn) and phosphatidylserine (PS or GPSer). Some glycerophospholipids in eukaryotic cells, such as phosphatidylinositols and phosphatidic acids are either precursors of, or are themselves, membrane-derived second messengers. Typically one or both of these hydroxyl groups are acylated with long-chain fatty acids, but there are also alkyl-linked and 1Z-alkenyl-linked (plasmalogen) glycerophospholipids, as well as dialkylether variants in prokaryotes.

Sphingolipids are a complex family of compounds that share a common structural feature, a sphingoid base backbone that is synthesized de novo from serine and a long-chain fatty acyl CoA, then converted into ceramides, phosphosphingolipids, glycosphingolipids and other species. The major sphingoid base of mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 14 to 26 carbon atoms. The major phosphosphingolipids of mammals are sphingomyelins (ceramide phosphocholines), whereas insects contain mainly ceramide phosphoethanolamines and fungi have phytoceramidephosphoinositols and mannose containing headgroups. The Glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives are an important component of membrane lipids, along with the glycerophospholipids and sphingomyelins. The steroids, which also contain the same fused four-ring core structure, have different biological roles as hormones and signaling molecules. The C18 steroids include the estrogen family whereas the C19 steroids comprise the androgens such as testosterone and androsterone. The C21 subclass includes the progestogens as well as the glucocorticoids and mineralocorticoids. The secosteroids, comprising various forms of vitamin D, are characterized by cleavage of the B ring of the core structure. Other examples of sterols are the bile acids and their conjugates.

Prenol lipids are synthesized from the 5-carbon precursors isopentenyl diphosphate and dimethylallyl diphosphate that are produced mainly via the mevalonic acid (MVA) pathway. The simple isoprenoids (linear alcohols, diphosphates, etc.) are formed by the successive addition of C5 units, and are classified according to number of these terpene units. Structures containing greater than 40 carbons are known as polyterpenes. Carotenoids are important simple isoprenoids that function as anti-oxidants and as precursors of vitamin A. Another biologically important class of molecules is exemplified by the quinones and hydroquinones, which contain an isoprenoid tail attached to a quinonoid core of non-isoprenoid origin. Vitamin E and vitamin K, as well as the ubiquinones, are examples of this class. Bacteria synthesize polyprenols (called bactoprenols) in which the terminal isoprenoid unit attached to oxygen remains unsaturated, whereas in animal polyprenols (dolichols) the terminal isoprenoid is reduced.

Saccharolipids describe compounds in which fatty acids are linked directly to a sugar backbone, forming structures that are compatible with membrane bilayers. In the saccharolipids, a sugar substitutes for the glycerol backbone that is present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo₂-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a very large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, and/or other processes. Many commonly used anti-microbial, anti-parasitic, and anti-cancer agents are polyketides or polyketide derivatives, such as erythromycins, tetracylines, avermectins, and antitumor epothilones.

The biomolecule of the present invention can be, but is not limited to, the lipids mentioned above.

In addition, the present invention relates to a paramagnetic biomolecule complex with more than one biomolecule. The more than one biomolecules can in one embodiment be any combination of the biomolecules mentioned herein.

The biomolecule moiety of the contrast agent may be a fragment of a biomolecule. Such fragments may be generated by any means, including hydrolysis, enzyme digestion and recombinant methods.

The one or more biomolecules of the invention, or the fragments thereof, may furthermore be modified by any means. One examples of modification is post-translational modification such as glycosylation of e.g. polypeptides.

Illustrative, non-limiting examples of biomolecules according to the present invention include globular proteins of from 1 kDa to preferably less than 1500 kDa, such as polypeptides comprising or consisting of aprotinin, chymtropsinogen A, lysozyme, ovalbumin, ribonucleases and cytochrome C, as well as fragments and/or variants thereof.

The invention in yet another embodiment comprises complexes wherein the biomolecule or biomolecules are genetically engineered. Said engineering can include recombinant production of the biomolecule or fragment or variant of the biomolecule

Further embodiments include biomolecules engineered so as to confer to the complex the ability to accumulate in the target compartment, or to enhance an integral ability of the biomolecule to do so. An example of such engineering includes introducing a binding site for Megalin and/or Cubilin to a biomolecule. A further example of modification is to engineer the biomolecule so as to change the electric charge of the biomolecule.

For example, the biomolecule moiety may be selected from the group comprising proteins capable of specific interaction with the Megalin/cubulin complex present in the proximal tubuli of the kidney. Complexes comprising such proteins, or fragments thereof, and which are thus retained in the kidney, are useful for use in visualising the kidney and processes in the kidney.

Megalin and Cubulin are also located in other organs/tissues besides the kidneys (see FIG. 13—from Christensen & Birn 2002). The present invention also relates to biomolecules engineered to accumulate in these organs/tissues.

In one embodiment of the invention, the one or more biomolecules of the invention comprise or consist of an aprotinin polypeptide.

In a further embodiment of the invention, the one or more biomolecules of the invention comprise or consist of a chymotropsinogen A polypeptide.

In another embodiment of the invention, the one or more biomolecules of the invention comprise or consist of a lysozyme polypeptide.

In a further embodiment of the invention, the one or more biomolecules of the invention comprise or consist of an ovalbumin polypeptide.

The biomolecule moiety of the novel contrast agent may furthermore comprise a fragment of a biomolecule, such as a polypeptide, for example a globular polypeptide, such as one or more fragments of aprotinin, chymtropsinogen A, lysozyme or ovalbumin.

The size of the biomolecule can influence the accumulation in the target compartment. The biomolecule of the invention preferably has a molecular weight of less than 1500 kDa.

A preferred embodiment has a molecular weight such as a MW in the range of from 1 kDa to 50 kDa, for example of from 1 kDa to 45 kDa, such as from 1 kDa to 40 kDa, for example of from 1 kDa to 35 kDa, such as from 1 kDa to 30 kDa, for example of from 1 kDa to 25 kDa, such as from 1 kDa to 24 kDa, for example of from 1 kDa to 23 kDa, such as from 1 kDa to 22 kDa, for example of from 1 kDa to 21 kDa, such as from 1 kDa to 20 kDa, for example of from 1 kDa to 19 kDa, such as from 1 kDa to 18 kDa, for example of from 1 kDa to 17 kDa, such as from 1 kDa to 16 kDa, for example of from 1 kDa to 15 kDa, such as from 1 kDa to 14 kDa, for example of from 1 kDa to 13 kDa, such as from 1 kDa to 12 kDa, for example of from 1 kDa to 11 kDa, such as from 1 kDa to 10 kDa, for example of from 1 kDa to 9 kDa, such as from 1 kDa to 8 kDa, for example of from 1 kDa to 7 kDa, such as from 1 kDa to 6 kDa, for example of from 1 kDa to 5 kDa, for example of from 2 kDa to 45 kDa, such as from 2 kDa to 40 kDa, for example of from 2 kDa to 35 kDa, such as from 2 kDa to 30 kDa, for example of from 2 kDa to 25 kDa, such as from 2 kDa to 24 kDa, for example of from 2 kDa to 23 kDa, such as from 2 kDa to 22 kDa, for example of from 2 kDa to 21 kDa, such as from 2 kDa to 20 kDa, for example of from 2 kDa to 19 kDa, such as from 2 kDa to 18 kDa, for example of from 2 kDa to 17 kDa, such as from 2 kDa to 16 kDa, for example of from 2 kDa to 15 kDa, such as from 2 kDa to 14 kDa, for example of from 2 kDa to 13 kDa, such as from 2 kDa to 12 kDa, for example of from 2 kDa to 11 kDa, such as from 2 kDa to 10 kDa, for example of from 2 kDa to 9 kDa, such as from 2 kDa to 8 kDa, for example of from 2 kDa to 7 kDa, such as from 2 kDa to 6 kDa, for example of from 2 kDa to 5 kDa, for example of from 5 kDa to 45 kDa, such as from 5 kDa to 40 kDa, for example of from 5 kDa to 35 kDa, such as from 5 kDa to 30 kDa, for example of from 5 kDa to 25 kDa, such as from 5 kDa to 24 kDa, for example of from 5 kDa to 23 kDa, such as from 5 kDa to 22 kDa, for example of from 5 kDa to 21 kDa, such as from 5 kDa to 20 kDa, for example of from 5 kDa to 19 kDa, such as from 5 kDa to 18 kDa, for example of from 5 kDa to 17 kDa, such as from 5 kDa to 16 kDa, for example of from 5 kDa to 15 kDa, such as from 5 kDa to 14 kDa, for example of from 5 kDa to 13 kDa, such as from 5 kDa to 12 kDa, for example of from 5 kDa to 11 kDa, such as from 5 kDa to 10 kDa, for example of from 5 kDa to 9 kDa, such as from 5 kDa to 8 kDa, for example of from 5 kDa to 7 kDa, such as from 5 kDa to 6 kDa, for example of from 10 kDa to 45 kDa, such as from 10 kDa to 40 kDa, for example of from 10 kDa to 35 kDa, such as from 10 kDa to 30 kDa, for example of from 10 kDa to 25 kDa, such as from 10 kDa to 24 kDa, for example of from 10 kDa to 23 kDa, such as from 10 kDa to 22 kDa, for example of from 10 kDa to 21 kDa, such as from 10 kDa to 20 kDa, for example of from 10 kDa to 19 kDa, such as from 10 kDa to 18 kDa, for example of from 10 kDa to 17 kDa, such as from 10 kDa to 16 kDa, for example of from 10 kDa to 15 kDa, such as from 10 kDa to 14 kDa, for example of from 10 kDa to 13 kDa, such as from 10 kDa to 12 kDa, for example of from 15 kDa to 45 kDa, such as from 15 kDa to 40 kDa, for example of from 15 kDa to 35 kDa, such as from 15 kDa to 30 kDa, for example of from 15 kDa to 25 kDa, such as from 15 kDa to 24 kDa, for example of from 15 kDa to 23 kDa, such as from 15 kDa to 22 kDa, for example of from 15 kDa to 21 kDa, such as from 15 kDa to 20 kDa, for example of from 15 kDa to 19 kDa, such as from 15 kDa to 18 kDa, for example of from 15 kDa to 17 kDa.

In one embodiment the biomolecule has a molecular weight in the range of from 1 kDa to 50 kDa, such as from 1 kDa to 2 kDa, for example from 2 kDa to 3 kDa, such as from 3 kDa to 4 kDa, for example from 4 kDa to 5 kDa, such as from 5 kDa to 6 kDa, for example from 6 kDa to 7 kDa, such as from 7 kDa to 8 kDa, for example from 8 kDa to 9 kDa, such as from 9 kDa to 10 kDa, for example from 10 kDa to 11 kDa, such as from 11 kDa to 12 kDa, for example from 12 kDa to 13 kDa, such as from 13 kDa to 14 kDa, for example from 14 kDa to 15 kDa, such as from 15 kDa to 16 kDa, for example from 16 kDa to 17 kDa, such as from 17 kDa to 18 kDa, for example from 18 kDa to 19 kDa, such as from 19 kDa to 20 kDa, for example from 20 kDa to 21 kDa, such as from 21 kDa to 22 kDa, for example from 22 kDa to 23 kDa, such as from 23 kDa to 24 kDa, for example from 24 kDa to 25 kDa, such as from 25 kDa to 26 kDa, for example from 26 kDa to 27 kDa, such as from 27 kDa to 28 kDa, for example from 28 kDa to 29 kDa, such as from 29 kDa to 30 kDa, for example from 30 kDa to 31 kDa, such as from 31 kDa to 32 kDa, for example from 32 kDa to 33 kDa, such as from 33 kDa to 34 kDa, for example from 34 kDa to 35 kDa, such as from 35 kDa to 36 kDa, for example from 36 kDa to 37 kDa, such as from 37 kDa to 38 kDa, for example from 38 kDa to 39 kDa, such as from 39 kDa to 40 kDa, for example from 40 kDa to 41 kDa, such as from 41 kDa to 42 kDa, for example from 42 kDa to 43 kDa, such as from 43 kDa to 44 kDa, for example from 44 kDa to 45 kDa, such as from 45 kDa to 46 kDa, for example from 46 kDa to 47 kDa, such as from 47 kDa to 48 kDa, for example from 48 kDa to 49 kDa, such as from 49 kDa to 50 kDa.

The biomolecule of the invention is not limited by the pI. The one or more biomolecules of the invention preferably have an isoelectric point in the range of from 4.5 to 11.5, such as from 4.5 to 11, for example of from 4.5 to 10.5, such as from 4.5 to 10, for example of from 4.5 to 9.5, such as from 4.5 to 9, for example of from 4.5 to 8.5, such as from 4.5 to 8, for example of from 4.5 to 7.5, such as from 4.5 to 7, for example of from 4.5 to 6.5, such as from 4.5 to 6, for example of from 4.5 to 5.5, such as from 4.5 to 5; for example of 5 to 11.5, such as from 5 to 11, for example of from 5 to 10.5, such as from 5 to 10, for example of from 5 to 9.5, such as from 5 to 9, for example of from 5 to 8.5, such as from 5 to 8, for example of from 5 to 7.5, such as from 5 to 7, for example of from 5 to 6.5, such as from 5 to 6, for example of from 5 to 5.5; for example of 5.5 to 11.5, such as from 5.5 to 11, for example of from 5.5 to 10.5, such as from 5.5 to 10, for example of from 5.5 to 9.5, such as from 5.5 to 9, for example of from 5.5 to 8.5, such as from 5.5 to 8, for example of from 5.5 to 7.5, such as from 5.5 to 7, for example of from 5.5 to 6.5, such as from 5.5 to 6; 6 to 11.5, such as from 6 to 11, for example of from 6 to 10.5, such as from 6 to 10, for example of from 6 to 9.5, such as from 6 to 9, for example of from 6 to 8.5, such as from 6 to 8, for example of from 6 to 7.5, such as from 6 to 7, for example of from 6 to 6.5; 6.5 to 11.5, such as from 6.5 to 11, for example of from 6.5 to 10.5, such as from 6.5 to 10, for example of from 6.5 to 9.5, such as from 6.5 to 9, for example of from 6.5 to 8.5, such as from 6.5 to 8, for example of from 6.5 to 7.5, such as from 6.5 to 7; for example of 7 to 11.5, such as from 7 to 11, for example of from 7 to 10.5, such as from 7 to 10, for example of from 7 to 9.5, such as from 7 to 9, for example of from 7 to 8.5, such as from 7 to 8, for example of from 7 to 7.5; for example of 7.5 to 11.5, such as from 7.5 to 11, for example of from 7.5 to 10.5, such as from 7.5 to 10, for example of from 7.5 to 9.5, such as from 7.5 to 9, for example of from 7.5 to 8.5, such as from 7.5 to 8; 8 to 11.5, such as from 8 to 11, for example of from 8 to 10.5, such as from 8 to 10, for example of from 8 to 9.5, such as from 8 to 9, for example of from 8 to 8.5; 8.5 to 11.5, such as from 8.5 to 11, for example of from 8.5 to 10.5, such as from 8.5 to 10, for example of from 8.5 to 9.5, such as from 8.5 to 9; for example of 9 to 11.5, such as from 9 to 11, for example of from 9 to 10.5, such as from 9 to 10, for example of from 9 to 9.5; for example of 9.5 to 11.5, such as from 9.5 to 11, for example of from 9.5 to 10.5, such as from 9.5 to 10; for example of 10 to 11.5, such as from 10 to 11, for example of from 10 to 10.5; for example of 10.5 to 11.5, such as from 10.5 to 11, for example from 11 to 11.5.

In one embodiment the biomolecule has an isolelectric point in the range of from 4.5 to 11.5, such as from 4.5 to 4.6, for example from 4.6 to 4.8, such as from 4.8 to 5.0, for example from 5.0 to 5.2, such as from 5.2 to 5.4, for example from 5.4 to 5.6, such as from 5.6 to 5.8, for example from 5.8 to 6.0, such as from 6.0 to 6.2, for example from 6.2 to 6.4, such as from 6.4 to 6.6, for example from 6.6 to 6.8, such as from 6.8 to 7.0, for example from 7.0 to 7.2, such as from 7.2 to 7.4, for example from 7.4 to 7.6, such as from 7.6 to 7.8, for example from 7.8 to 8.0, such as from 8.0 to 8.2, for example from 8.2 to 8.4, such as from 8.4 to 8.6, for example from 8.6 to 8.8, such as from 8.8 to 9.0, for example from 9.0 to 9.2, such as from 9.2 to 9.4, for example from 9.4 to 9.6, such as from 9.6 to 9.8, for example from 9.8 to 10.0, such as from 10.0 to 10.2, for example from 10.2 to 10.4, such as from 10.4 to 10.6, for example from 10.6 to 10.8, such as from 10.8 to 11.0, for example from 11.0 to 11.2, such as from 11.2 to 11.4, for example from 11.4 to 11.5.

The complexes per se, and uses of the complexes, are further described and exemplified below.

### The Marker Moiety

The marker moiety of the complex may be any marker useful for visualising a target compartment. The marker may be for example fluorescent, paramagnetic or radioactive. Prefereably, the marker is selected from the group containing paramagnetic markers and radioactive markers. A paramagnetic marker is any chemical or substance which provides a paramagnetic signal useful in imaging methods. A radioactive marker comprises or consists of a substance which gives off, or is capable of giving off, radiant energy in the form of particles (alpha or beta radiation) or rays (gamma radiation) by the spontaneous disintegration of the nuclei of atoms.

The invention in one embodiment is directed to complexes comprising one or more paramagnetic markers. The paramagnetic marker may for example be one or more of the paramagnetic isotopes of Aluminium, Barium, Calcium, Dysprosium, Gadolinium, Magnesium, Manganese, Oxygen, Platinum, Sodium, Strontium, Technetium and Uranium or any variant thereof and/or mixtures thereof.

The paramagnetic marker is preferably a magnetic moment greater than 6, more preferably around 7. One example of such a paramagnetic marker is Gadolinium.

The marker moiety of the complex may also be selected from the group consisting of radioactive markers, more preferably from the group of radioactive markers which are tolerable to a living individual. The radioactive marker may be, for example, radioactive Technetium, a radioactive, iodinated species, or a substance comprising radioactive isotopes of Carbon.

In one embodiment the marker molecule has a molecular weight in the range of from 1 kDa to 50 kDa, such as from 1 kDa to 2 kDa, for example from 2 kDa to 3 kDa, such as from 3 kDa to 4 kDa, for example from 4 kDa to 5 kDa, such as from 5 kDa to 6 kDa, for example from 6 kDa to 7 kDa, such as from 7 kDa to 8 kDa, for example from 8 kDa to 9 kDa, such as from 9 kDa to 10 kDa, for example from 10 kDa to 11 kDa, such as from 11 kDa to 12 kDa, for example from 12 kDa to 13 kDa, such as from 13 kDa to 14 kDa, for example from 14 kDa to 15 kDa, such as from 15 kDa to 16 kDa, for example from 16 kDa to 17 kDa, such as from 17 kDa to 18 kDa, for example from 18 kDa to 19 kDa, such as from 19 kDa to 20 kDa, for example from 20 kDa to 21 kDa, such as from 21 kDa to 22 kDa, for example from 22 kDa to 23 kDa, such as from 23 kDa to 24 kDa, for example from 24 kDa to 25 kDa, such as from 25 kDa to 26 kDa, for example from 26 kDa to 27 kDa, such as from 27 kDa to 28 kDa, for example from 28 kDa to 29 kDa, such as from 29 kDa to 30 kDa, for example from 30 kDa to 31 kDa, such as from 31 kDa to 32 kDa, for example from 32 kDa to 33 kDa, such as from 33 kDa to 34 kDa, for example from 34 kDa to 35 kDa, such as from 35 kDa to 36 kDa, for example from 36 kDa to 37 kDa, such as from 37 kDa to 38 kDa, for example from 38 kDa to 39 kDa, such as from 39 kDa to 40 kDa, for example from 40 kDa to 41 kDa, such as from 41 kDa to 42 kDa, for example from 42 kDa to 43 kDa, such as from 43 kDa to 44 kDa, for example from 44 kDa to 45 kDa, such as from 45 kDa to 46 kDa, for example from 46 kDa to 47 kDa, such as from 47 kDa to 48 kDa, for example from 48 kDa to 49 kDa, such as from 49 kDa to 50 kDa.

In one embodiment the marker molecule has an isolelectric point in the range of from 4.5 to 11.5, such as from 4.5 to 4.6, for example from 4.6 to 4.8, such as from 4.8 to 5.0, for example from 5.0 to 5.2, such as from 5.2 to 5.4, for example from 5.4 to 5.6, such as from 5.6 to 5.8, for example from 5.8 to 6.0, such as from 6.0 to 6.2, for example from 6.2 to 6.4, such as from 6.4 to 6.6, for example from 6.6 to 6.8, such as from 6.8 to 7.0, for example from 7.0 to 7.2, such as from 7.2 to 7.4, for example from 7.4 to 7.6, such as from 7.6 to 7.8, for example from 7.8 to 8.0, such as from 8.0 to 8.2, for example from 8.2 to 8.4, such as from 8.4 to 8.6, for example from 8.6 to 8.8, such as from 8.8 to 9.0, for example from 9.0 to 9.2, such as from 9.2 to 9.4, for example from 9.4 to 9.6, such as from 9.6 to 9.8, for example from 9.8 to 10.0, such as from 10.0 to 10.2, for example from 10.2 to 10.4, such as from 10.4 to 10.6, for example from 10.6 to 10.8, such as from 10.8 to 11.0, for example from 11.0 to 11.2, such as from 11.2 to 11.4, for example from 11.4 to 11.5.

In one embodiment the marker molecule is a fluorescent marker molecule. The fluorescent marker molecule can be selected from the group consisting of 5-(and 6)-carboxyfluorescein, 5- or 6-carboxyfluorescein, 6-(fluorescein)-5-(and 6)-carboxamido hexanoic acid, fluorescein isothiocyanate (FITC), rhodamine, tetramethylrhodamine, optionally substituted coumarin including AMCA, PerCP, phycobiliproteins including R-phycoerythrin (RPE) and allophycoerythrin (APC), Texas Red, Princeston Red, Green fluorescent protein (GFP) and analogues thereof, and conjugates of R-phycoerythrin or allophycoerythrin or Texas Red, and inorganic fluorescent labels based on semiconductor nanocrystals (like quantum dot and Qdot™ nanocrystals), and time-resolved fluorescent labels based on lanthanides like Eu3+ and Sm3+, Fluor dyes, Pacific Blue™, Pacific Orange™, Cascade Yellow™, AlexaFluor® (AF), AF405, AF488, AF500, AF514, AF532, AF546, AF555, AF568, AF594, AF610, AF633, AF635, AF647, AF680, AF700, AF710, AF750, AF800, Quantum Dot based dyes, QDot® Nanocrystals (Invitrogen, MolecularProbs), Qdot®525, Qdot®565, Qdot®585, Qdot®605, Qdot®655, Qdot®705, Qdot®800, DyLight™ Dyes (Pierce) (DL), DL549, DL649, DL680, DL800, Fluorescein (Flu) or any derivate thereof, Cy-Dyes, Cy2, Cy3, Cy3.5, Cy5, Cy5.5, Cy7, Fluorescent Proteins, RPE, PerCp, APC, Green fluorescent proteins; GFP and GFP derivated mutant proteins; BFP, CFP, YFP, DsRed, T1, Dimer2, mRFP1, MBanana, mOrange, dTomato, tdTomato, mTangerine, mStrawberry, mCherry, Tandem dyes, RPE-Cy5, RPE-Cy5.5, RPE-Cy7, RPE-AlexaFluor® tandem conjugates; RPE-Alexa610, RPE-TxRed, APC-Aleca600, APC-Alexa610, APC-Alexa750, APC-Cy5, APC-Cy5.5, Ionophors; ion chelating fluorescent props

The tables below list further examples of marker molecules.

| | Excitation | Emission |
|---|---|---|
| Fluorophor/Fluorochrome | nm | nm |
| 2-(4′-maleimidylanilino)naphthalene-6-sulfonic | 322 | 417 |
| acid, sodium salt | | |
| 5-((((2-iodoacetyl)amino)ethyl)amino) | 336 | 490 |
| naphthalene-1-sulfonic acid | | |
| Pyrene-1-butanoic acid | 340 | 376 |
| AlexaFluor 350 (7-amino-6-sulfonic acid-4- | 346 | 442 |
| methyl coumarin-3-acetic acid) | | |
| AMCA (7-amino-4-methyl coumarin-3-acetic | 353 | 442 |
| acid) | | |
| 7-hydroxy-4-methyl coumarin-3-acetic acid | 360 | 455 |
| Marina Blue (6,8-difluoro-7-hydroxy-4-methyl | 362 | 459 |
| coumarin-3-acetic acid) | | |
| 7-dimethylamino-coumarin-4-acetic acid | 370 | 459 |
| Fluorescamin-N-butyl amine adduct | 380 | 464 |
| 7-hydroxy-coumarine-3-carboxylic acid | 386 | 448 |
| CascadeBlue (pyrene-trisulphonic acid acetyl | 396 | 410 |
| azide) | | |
| Pacific Blue (6,8 difluoro-7-hydroxy coumarin-3- | 416 | 451 |
| carboxylic acid) | | |
| 7-diethylamino-coumarin-3-carboxylic acid | 420 | 468 |
| N-(((4-azidobenzoyl)amino)ethyl)-4-amino-3,6- | 426 | 534 |
| disulfo-1,8-naphthalimide, dipotassium salt | | |
| Alexa Fluor 430 | 434 | 539 |
| 3-perylenedodecanoic acid | 440 | 448 |
| 8-hydroxypyrene-1,3,6-trisulfonic acid, trisodium | 454 | 511 |
| salt | | |
| 12-(N-(7-nitrobenz-2-oxa-1,3-diazol-4- | 467 | 536 |
| yl)amino)dodecanoic acid | | |
| N,N′-dimethyl-N-(iodoacetyl)-N′-(7-nitrobenz-2- | 478 | 541 |
| oxa-1,3-diazol-4-yl)ethylenediamine | | |
| Oregon Green 488 (difluoro carboxy fluorescein) | 488 | 518 |
| 5-iodoacetamidofluorescein | 492 | 515 |
| propidium iodide-DNA adduct | 493 | 636 |
| Carboxy fluorescein | 495 | 519 |

| Fluorochrome family | Example fluorochrome |
|---|---|
| AlexaFluor ®(AF) | AF ® 350, AF405, AF430, AF488, AF500, AF514, AF532, AF546, |
| | AF555, AF568, AF594, AF610, AF633, AF635, AF647, AF680, |
| | AF700, AF710, AF750, AF800 |
| Quantum Dot (Qdot ®) based | Qdot ® 525, Qdot ® 565, Qdot ® 585, Qdot ® 605, Qdot ® 655, Qdot ® 705, |
| dyes | Qdot ® 800 |
| DyLight ™ Dyes (DL) | DL549, DL649, DL680, DL800 |
| Small fluorescing dyes | FITC, Pacific Blue ™, Pacific Orange ™, Cascade Yellow ™, Marina |
| | blue ™, DSred, DSred-2, 7-AAD, TO-Pro-3, |
| Cy-Dyes | Cy2, Cy3, Cy3.5, Cy5, Cy5.5, Cy7 |
| Phycobili Proteins: | R-Phycoerythrin (RPE), PerCP, Allophycocyanin (APC), B- |
| | Phycoerythrin, C-Phycocyanin |
| Fluorescent Proteins | (E)GFP and GFP ((enhanced) green fluorescent protein) derived |
| | mutant proteins; BFP, CFP, YFP, DsRed, T1, Dimer2, |
| | mRFP1, MBanana, mOrange, dTomato, tdTomato, mTangerine, |
| | mStrawberry, mCherry |
| Tandem dyes with RPE | RPE-Cy5, RPE-Cy5.5, RPE-Cy7, RPE-AlexaFluor ® tandem |
| | conjugates; RPE-Alexa610, RPE-TxRed |
| Tandem dyes with APC | APC-Aleca600, APC-Alexa610, APC-Alexa750, APC-Cy5, APC- |
| | Cy5.5 |
| Calcium dyes | Indo-1-Ca2+ Indo-2-Ca2+ |

### Linkage

The third step comprises linking the biomolecule to the marker moiety, optionally via a linker.

The link between the biomolecules and the markers may be direct or indirect. The nature of the indirect or direct linkage may be of a single nature or comprise several species of binding, e.g. comprise both covalent binding and ionic interactions.

In one embodiment comprising indirect linkage, the biomolecule is linked to a metal ion chelator, and the marker is chelated by said chelator.

Examples of chelators are: diethylenetriaminepentaacetic acid (DTPA), diethylenetriamine-pentamethylenephosphonic acid (DTPMP), tetraazacyclododecanetetraacetic acid (DOTA) or a derivative of DOTA, ethylene-diaminetetraacetic acid (EDTA), tetraazacyclododecanetetrakis (methylene phosphonic acid) (DOTP), hydroxypropyl tetraazacylododecanetriacetic acid (HP-DO3A), diethylenetriaminetriacetic acid bismethylamide (DTPA-BMA) and MS-325.

Depending on the conditions of the coupling reaction, the single chelator can link together two or more biomolecule complexes (B-X-M) to form supracomplexes, which are designated here without prejudice as (B-X-M)ⁿ, n=a positive integer. The term contrast agents comprises both sets of complexes. In another embodiment the biomolecule complex can comprise Bⁿ—Xⁿ-Mⁿ, wherein n is a positive integer such as 1, 2, 3, 4, 5, 6, 7, 8, 9, 10 etc.

In one embodiment of the present invention, one or more paramagnetic markers associated with one or more biomolecules form a complex comprising a linker moiety associating or binding the one or more paramagnetic markers and the one or more biomolecule(s).

The conditions of the coupling reaction can be manipulated so as to yield different subpopulations of complexes, where the size and charge (pI) will vary between the subpopulations.

The present invention also encompasses complexes comprising more that one different biomolecules and markers. These may be sythesized simultaneously in the same reaction mix, or synthesised separately and mixed subsequently at any stage.

The invention may for example comprise a chelator and a chelated globular protein. For example, the chelator may be DTPA, and the globular protein may be one of the group comprising

The chemical reaction between aprotinin and Gd-DTPA serves as one illustrative example. Suitable reaction conditions yield several subpopulations of Gd-DTPA-Aprotinin complexes with decreasing pI corresponding to the amount of incorporated Gd-DTPA. In this example, a very small fraction to the right remains unincorporated and co-elutes with native aprotinin. Anionic complexes of gdAp are also produced in this reaction and are seen in the flow-through prior to the salt gradient. These are the most heavily incorporated with Gd-DTPA of the subpopulations.

Appropriate molecules, capable of providing non covalent interactions between the marker molecule and the biomolecule, involve the following molecule pairs and molecules: streptavidin/biotin, avidin/biotin, antibody/antigen, DNA/DNA, DNA/PNA, DNA/RNA, PNA/PNA, LNA/DNA, leucine zipper e.g. Fos/Jun, IgG dimeric protein, IgM multivalent protein, acid/base coiled-coil helices, chelate/metal ion-bound chelate, streptavidin (SA) and avidin and derivatives thereof, biotin, immunoglobulins, antibodies (monoclonal, polyclonal, and recombinant), antibody fragments and derivatives thereof, leucine zipper domain of AP-1 (jun and fos), hexa-his (metal chelate moiety), hexa-hat GST (glutathione S-transferase) glutathione affinity, Calmodulin-binding peptide (CBP), Strep-tag, Cellulose Binding Domain, Maltose Binding Protein, S-Peptide Tag, Chitin Binding Tag, Immuno-reactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, VSV Epitope, lectins that mediate binding to a diversity of compounds, including carbohydrates, lipids and proteins, e.g. Con A (Canavalia ensiformis) or WGA (wheat germ agglutinin) and tetranectin or Protein A or G (antibody affinity). Combinations of such binding entities are also comprised.

### Selecting the Complex

The molecules of desired charge and size are purified and used as a contrast agent in imaging methods.

Methods of separation are well-known in the art. The preferred methods of separation include separation by size, and by charge. The selection may for example be performed by gel chromatography or ion exchange chromatography.

### Complexes of the Invention

The present invention relates to novel contrast agents in the form of complexes comprising one or more biomolecules linked to one or more markers, including contrast agents obtainable by the methods described above.

Complexes according to the present invention may be illustrated by the general formula B-X-M, wherein B is indicative of one or more biomolecules, X is an optional linker moiety, for example a chelator, and M is a marker moiety. M may for example be a paramagnetic moiety, a radioactive label or a fluorescent label.

The one or more biomolecules and the one or more markers which are part of the complexes are discussed in more detail above.

The one or more markers which are part of the complexes are discussed in more detail above.

The complexes according to the invention are preferably differentially accumulated in a target compartment. The contrast agents are useful in imaging techniques for visualising target compartments in an individual.

The methods of making the complexes make it possible to tailor complexes for use in visualising specific target compartments (see above).

In one embodiment, the complex according to the invention comprises Gadolinium as the at least one paramagnetic marker. In a further embodiment, the complex according to the invention comprises aprotinin linked to Gadolinium. Another embodiment of the invention is directed to a complex comprising chymotrypsinogen A linked to

Gadolinium. A still further embodiment of the invention is directed to a complex comprising lysozyme linked to Gadolinium. In yet another embodiment the invention is directed to a complex comprising ovalbumin linked to Gadolinium.

### Methods of Using Complexes

The complexes are useful for both static and dynamic imaging processes. One example of a dynamic imaging process is visualising the glomerular filtration rate in the kidney. One example of a static imaging is use of the complexes in CT-scans to localise the presence of cancer tumors.

The present invention in further embodiments is directed to magnetic resonance imaging (MRI) based methods wherein complexes according to the invention, or contrast agents comprising such complexes, are used. In particular, there is provided a magnetic resonance imaging method for the measurement of the glomerular filtration rate of the kidney in an individual, the method comprising the steps of administering to a subject the complex according to the invention, capturing images of the kidney of said subject and calculating the glomerular filtration rate on the basis of the captured images.

Examples of visualisation techniques comprise magnetic resonance imaging and radiographic methods such as computer tomography-based imaging methods.

The complexes of the inventions can be used for functional assessment of an organ, such as functional assessment of a kidney in an individual, such as a human being, functional assessment of a small intestine in an individual, such as a human being, and assessment of the vasculature in an individual, such as a human being.

In the example of the kidney, the accumulation in the kidney enables sensitive and high resolution scans of the kidney to be performed. This in turn renders possible quantitative assessment of the GFR. Moreover, since a proteinuric kidney displays characteristic MRI scans depending on the source of proteinuria, the methods of the invention also makes it possible to gather detailed information on the underlying cause of decreased GFR. Furthermore, information from single kidneys can be gained

In another embodiment the invention provides a method for functional assessment of the kidney by performing sequential MR imaging scans of the subject using complexes with different traits. Combination of data acquired from scans with different complexes of the invention gives detailed information on the type and scope of kidney damage/function.

The invention aslo relates to methods for performing MR imaging scans. Use of more than one complex preferably gives more information than can be gained by a single scan of a single complex.

The above-cited MR imaging based methods can be used for both diagnostic and non-diagnostic applications. Accordingly, in one aspect of the present invention there is provided a method for diagnosing individuals suffering from an abnormal glomerular filtration rate of the kidney.

In another embodiment there is provided a non-diagnostic MR imaging based method for the assessment of the glomerular filtration rate of the kidney in an individual in need thereof.

The complexes may be administered simultaneously, for example more than one complex may they can be present in a composition for use as a contrast agent. The complexes may also be administered sequentially in any order, wherein one complex is administered first and a second complex is administered after a certain time. This time interval may be from between 5 minutes and up to about 48 hours.

The biomolecule complexes of the present invention can be used for any MRI analysis including the ones mentioned below.
Brain Imaging: Stroke and tumor evaluation, vascular imaging, surgery planning, MRI Angiography or Venography studies, MRI Diffusion and Perfusion Imaging, Functional MRI, MRI Brain Spectroscopy.
Cardiac Imaging: RV dysplasia, Congenital, Tumor, Mass etc.
Vascular Imaging: Imaging and enhancing vessels with MRI Angiography.
Spine Imaging: Cervical, thoracic, lumbar and spine imaging for congenital abnormalities, cord compression, tumor, HNP, sciatica etc.
Abdomen/Pelvis Imaging: cancer diagnosing/staging, MRI or MRV Angiographic studies, Renal donor evaluations, Graft and Aneurysm evaluation
Orthopedic Imaging: Imaging of soft tissue, muscle ligaments, and bones

### Complexes for Use in MRI Imaging of Kidney

One example of uses for the complexes of the invention concerns use for visualising the glomerular filtration rate in the kidney.

Illustrative, non-limiting examples of biomolecules according to the present invention include globular proteins of from 1 kDa to preferably less than 500 kDa, such as polypeptides comprising or consisting of aprotinin, chymtropsinogen A, lysozyme, ovalbumin, ribonucleases and cytochrome C, as well as fragments and/or variants thereof.

In one embodiment of the invention, the one or more biomolecules of the invention comprise or consist of an aprotinin polypeptide.

In a further embodiment of the invention, the one or more biomolecules of the invention comprise or consist of a chymotropsinogen A polypeptide.

In another embodiment of the invention, the one or more biomolecules of the invention comprise or consist of a lysozyme polypeptide.

In a further embodiment of the invention, the one or more biomolecules of the invention comprise or consist of an ovalbumin polypeptide.

The biomolecule moiety of the novel contrast agent may furthermore comprise a fragment of a biomolecule, such as a polypeptide, for example a globular polypeptide, such as one or more fragments of aprotinin, chymtropsinogen A, lysozyme or ovalbumin.

The invention in yet another embodiment comprises complexes wherein the biomolecule or biomolecules are genetically engineered. Said engineering can include recombinant production of the biomolecule or fragment or variant of the biomolecule

Further particular embodiments include biomolecules engineered so as to confer to the complex the ability to accumulate in the kidney, or to enhance an integral ability of the biomolecule to do so. An example of such engineering includes introducing a binding site for Megalin and/or Cubilin to a biomolecule. A further example of modification is to engineer the biomolecule so as to change the electric charge of the biomolecule.

In one particular embodiment, the complex comprises aprotinin as a biomolecule. Aprotinin interacts specifically with the Megalin/Cubulin complex and is specifically resorbed by the proximal tubuli. The complex further comprises DPTA as a linker moiety. The complex is characterised by Gadolinium as the marker moiety.

In one example, provided in order to illustrate and not to limit, a complex comprising aprotinin linked to Gadolinium is administered intravenously to a patient and MR imaging performed. Imaging visualises the glomerular filtration rate of said patient. Subsequently, a complex comprising a lysozyme derivative linked to Gadolinium is administered intravenously to the same patient and MR imaging performed. The images acquired in the two scans are compared. Deficient uptake of complex comprising a lysozyme derivative (tubular injury inhibits absorption of this complex) in the absence of deficiency of the complex comprising aprotinin (absorbed even by injured tubules) indicates damage (e.g. after acute ischemia) locally to tubuli which has not yet resulted in lowered glomerular filtration rate.

### Administration

The modes of administration of the complexes or pharmaceutical compositions comprising the complexes of the invention may be enteral or parenteral, for example orally, sublingually, nasally, by inhalation, injection, intravenously, subcutaneously, rectally, vaginally, cutaneously or transdermally. In preferred embodiments the complexes are administered perorally or intravenously. Compositions comprising or consisting of the complexes may also be administered by enema.

One preferred mode of adminstering of the complexes or pharmaceutical compositions comprising the complexes of the invention is intravenously. Examples of intravenous administration include, but are not limited to, injection or via gravity drip. The administration may for example be a bolus injection. Another example of mode of administration may be a short infusion, for example for 1 minute, such as for 2 to 3 minutes, for example for 4 minutes, such as for about 5 minutes, for example for about 6 minutes, such as for about 7 minutes, for example for about 8 minutes, such as for about 9 minutes, for example for about 10 minutes, such as for about 12 minutes, for example for about 15 minutes, such as for about 16 minutes, for example for about 17 minutes, such as for about 18 minutes, for example for about 19 minutes, such as for about 20 minutes

Another preferred mode of administering to an individual of the complexes and compositions according to the invention is perorally. The preferred dosage form can be a solution, a tonic, a tablet, a capsule, a lozenge, a chewable tablet, and the like.

The dosis of complexes comprise a safe and sufficient amount of the complexes, which is preferably in the range of from about 0.01 mg to about 200 mg per dosis. The dosis may be for example from 1 mg to 10 mg, for example from 1 mg to 9 mg, such as from 1 mg to 8 mg, for example from 1 mg to 7 mg, such as from 1 mg to 6 mg, for example from 1 mg to 5 mg, such as from 1 mg to 4 mg, for example from 1 mg to 3 mg, such as from 1 mg to 2 mg, for example from 2 mg to 10 mg, such as from 2 mg to 9 mg, for example from 2 mg to 8 mg, such as from 2 mg to 7 mg, for example from 2 mg 6 mg, such as from 2 mg to 5 mg, for example from 2 mg to 4 mg, such as from 2 mg to 3 mg, for example from 3 mg to 10 mg, or example from 3 mg to 9 mg, such as from 3 mg to 8 mg, for example from 3 mg to 7 mg, such as from 3 mg to 6 mg, for example from 3 mg to 5 mg, such as from 3 mg to 4 mg, for example from 4 mg to 10 mg, such as from 4 mg to 9 mg, for example from 4 mg to 8 mg, such as from 4 mg to 7 mg, for example from 4 mg to 6 mg, such as from 4 mg to 5 mg, for example from 5 mg to 10 mg, such as from 5 mg to 9 mg, for example from 5 mg to 8 mg, such as from 5 mg to 7 mg, for example from 5 to 6 mg, for example from 6 mg to 10 mg, such as from 6 mg to 9 mg, for example from 6 mg to 8 mg, such as from 6 mg to 7 mg, for example from 7 mg to 10 mg, such as from 7 mg to 9 mg, for example from 7 mg to 8 mg, for example from 8 mg to 10 mg, such as from 8 mg to 9 mg, for example from 9 mg to 10 mg, or from 10 mg to 200 mg, for example from 10 mg to 190 mg, such as from 10 mg to 180 mg, for example from 10 mg to 170 mg, such as from 10 mg to 160 mg, for example from 10 mg to 150 mg, such as from 10 mg to 140 mg, for example from 10 mg to 130 mg, such as from 10 mg to 120 mg, for example from 10 mg to 110 mg, such as from 10 mg to 100 mg, for example from 10 mg to 90 mg, such as from 10 mg to 80 mg, for example from 10 mg to 70 mg, such as from 10 mg to 60 mg, for example from 10 mg to 50 mg, such as from 10 mg to 40 mg, for example from 10 mg to 30 mg, such as from 10 mg to 20 mg, such as from 20 mg to 200 mg, for example from 20 mg to 190 mg, such as from 20 mg to 180 mg, for example from 20 to 170 mg, such as from 20 to 160 mg, for example from 20 mg to 150 mg, such as from 20 mg to 140 mg, for example from 20 mg to 130 mg, such as from 20 mg to 120 mg, for example from 20 mg to 110 mg, such as from 20 mg to 100 mg, for example from 20 mg to 90 mg, such as from 20 mg to 80 mg, for example from 20 mg to 70 mg, such as from 20 mg to 60 mg, for example from 20 mg to 50 mg, such as from 20 mg to 40 mg, for example from 20 mg to 30 mg, such as from 30 mg to 200 mg, for example from 30 mg to 190 mg, such as from 30 mg to 180 mg, for example from 30 to 170 mg, such as from 30 to 160 mg, for example from 30 mg to 150 mg, such as from 30 mg to 140 mg, for example from 30 mg to 130 mg, such as from 30 mg to 120 mg, for example from 30 mg to 110 mg, such as from 30 mg to 100 mg, for example from 30 mg to 90 mg, such as from 30 mg to 80 mg, for example from 30 mg to 70 mg, such as from 30 mg to 60 mg, for example from 30 mg to 50 mg, such as from 30 mg to 40 mg, such as from 40 mg to 200 mg, for example from 40 mg to 190 mg, such as from 40 mg to 180 mg, for example from 40 to 170 mg, such as from 40 to 160 mg, for example from 40 mg to 150 mg, such as from 40 mg to 140 mg, for example from 40 mg to 130 mg, such as from 40 mg to 120 mg, for example from 40 mg to 110 mg, such as from 40 mg to 100 mg, for example from 40 mg to 90 mg, such as from 40 mg to 80 mg, for example from 40 mg to 70 mg, such as from 40 mg to 60 mg, for example from 40 mg to 50 mg, such as from 50 mg to 200 mg, for example from 50 mg to 190 mg, such as from 50 mg to 180 mg, for example from 50 to 170 mg, such as from 50 to 160 mg, for example from 50 mg to 150 mg, such as from 50 mg to 140 mg, for example from 50 mg to 130 mg, such as from 50 mg to 120 mg, for example from 50 mg to 110 mg, such as from 50 mg to 100 mg, for example from 50 mg to 90 mg, such as from 50 mg to 80 mg, for example from 50 mg to 70 mg, such as from 50 mg to 60 mg, such as from 60 mg to 200 mg, for example from 60 mg to 190 mg, such as from 60 mg to 180 mg, for example from 60 to 170 mg, such as from 60 to 160 mg, for example from 60 mg to 150 mg, such as from 60 mg to 140 mg, for example from 60 mg to 130 mg, such as from 60 mg to 120 mg, for example from 60 mg to 110 mg, such as from 60 mg to 100 mg, for example from 60 mg to 90 mg, such as from 60 mg to 80 mg, for example from 60 mg to 70 mg, such as from 70 mg to 200 mg, for example from 70 mg to 190 mg, such as from 70 mg to 180 mg, for example from 70 to 170 mg, such as from 70 to 160 mg, for example from 70 mg to 150 mg, such as from 70 mg to 140 mg, for example from 70 mg to 130 mg, such as from 70 mg to 120 mg, for example from 70 mg to 110 mg, such as from 70 mg to 100 mg, for example from 70 mg to 90 mg, such as from 70 mg to 80 mg, such as from 80 mg to 200 mg, for example from 80 mg to 190 mg, such as from 80 mg to 180 mg, for example from 80 to 170 mg, such as from 80 to 160 mg, for example from 80 mg to 150 mg, such as from 80 mg to 140 mg, for example from 80 mg to 130 mg, such as from 80 mg to 120 mg, for example from 80 mg to 110 mg, such as from 80 mg to 100 mg, for example from 80 mg to 90 mg, such as from 90 mg to 200 mg, for example from 90 mg to 190 mg, such as from 90 mg to 180 mg, for example from 90 to 170 mg, such as from 90 to 160 mg, for example from 90 mg to 150 mg, such as from 90 mg to 140 mg, for example from 90 mg to 130 mg, such as from 90 mg to 120 mg, for example from 90 mg to 110 mg, such as from 90 mg to 100 mg, such as from 100 mg to 200 mg, for example from 100 mg to 190 mg, such as from 100 mg to 180 mg, for example from 100 to 170 mg, such as from 100 to 160 mg, for example from 100 mg to 150 mg, such as from 100 mg to 140 mg, for example from 100 mg to 130 mg, such as from 100 mg to 120 mg, for example from 100 mg to 110 mg, such as from 110 mg to 200 mg, for example from 110 mg to 190 mg, such as from 110 mg to 180 mg, for example from 110 to 170 mg, such as from 110 to 160 mg, for example from 110 mg to 150 mg, such as from 110 mg to 140 mg, for example from 110 mg to 130 mg, such as from 110 mg to 120 mg, such as from 120 mg to 200 mg, for example from 120 mg to 190 mg, such as from 120 mg to 180 mg, for example from 120 to 170 mg, such as from 120 to 160 mg, for example from 120 mg to 150 mg, such as from 120 mg to 140 mg, for example from 120 mg to 130 mg, such as from 130 mg to 200 mg, for example from 130 mg to 190 mg, such as from 130 mg to 180 mg, for example from 130 to 170 mg, such as from 130 to 160 mg, for example from 130 mg to 150 mg, such as from 130 mg to 140 mg, such as from 140 mg to 200 mg, for example from 140 mg to 190 mg, such as from 140 mg to 180 mg, for example from 140 to 170 mg, such as from 140 to 160 mg, for example from 140 mg to 150 mg, such as from 150 mg to 200 mg, for example from 150 mg to 190 mg, such as from 150 mg to 180 mg, for example from 150 to 170 mg, such as from 150 to 160 mg, such as from 160 mg to 200 mg, for example from 160 mg to 190 mg, such as from 160 mg to 180 mg, for example from 160 to 170 mg, such as from 170 mg to 200 mg, for example from 170 mg to 190 mg, such as from 170 mg to 180 mg, such as from 180 mg to 200 mg, for example from 180 mg to 190 mg, such as from 190 mg to 200 mg,

Carriers suitable for the preparation of unit dosage forms for peroral administration are well-known in the art.

Peroral compositions also include liquid solutions, emulsions, suspensions, and the like. The carriers suitable for preparation of such compositions are well known in the art. Liquid oral compositions preferably comprise from about 0.001% to about 5% of the subject compound.

Other compositions useful for attaining systemic delivery of the complexes include sublingual and buccal dosage forms. Such compositions typically comprise soluble filler substances; and binders, as well as optional glidants, lubricants, sweeteners, colorants, antioxidants and flavoring agents may also be included.

### Combinations of Methods

The biomolecule complexes of the present invention can further be used for one or more of the imaging analysis described herein below or any combination thereof. The present invention is also directed to combinations of methods, for example CT-PET.

Positron emission tomography—computed tomography (also know as PET-CT) is a medical imaging device which combines in a single gantry system both a Positron Emission Tomography (PET) and an x-ray Computed Tomography, so that images acquired from both devices can be taken sequentially, in the same session from the patient and combined into a single superposed (co-registered) image. Thus, functional imaging obtained by PET, which depicts the spatial distribution of metabolic or biochemical activity in the body can be more precisely aligned or correlated with anatomic imaging obtained by CT scanning. Two- and three-dimensional image reconstruction may be rendered as a function of a common software and control system.

The biomolecule complex according to the present invention can be used for PET-CT analysis for example, in oncology, surgical planning, radiation therapy and cancer staging.

USG (ultrasonography), CT and MRI provide excellent mapping of e.g. morphology and extent of tumors. Recent advances in CT/MR perfusion, MR spectroscopy, Functional MR add certain information, even of metabolic activity. PET done with FDG gives both qualitative and quantitative metabolic information, which is very useful for early diagnosis and follow-up. Historically US/CT/MRI have been the domain of radiologists while SPECT, PET are managed by nuclear medicine colleagues.

MR spectroscopy is highly sensitive and specific e.g. in tumour detection, as high choline peaks are seen in cellular metaplasia. Both pyruvate and lactate can be easily detected on MRS, helping to grade the tumour and assess tumor activity.

Both MR and CT perfusion studies have been useful in evaluating viable tumor tissue, which normally shows increased perfusion. CT perfusion is further beneficial as it can quantify blood volume, blood flow and tumor transit time directly. This is of great help in follow up of tumors on different therapeutic regimes.

PET has poor anatomic detail and needs correlation with other imaging tools like CT to accurately localize the lesion and to differentiate normal from abnormal tracer uptake. In one embodiment high resolution CT of desired organ is obtained with superimposition of PET images on underlying anatomical data, leading to unparalleled imaging acquisition.

CT-PET is well established in diagnosis, staging and follow-up of e.g. colorectal cancer, oesophageal malignancies, lymphomas, lung cancer, melanomas, breast malignancy, head and neck tumors and in characterisation of a pulmonary nodule. In addition, CT-PET has been found invaluable in accurate localization of very small areas of increased traced activity.

### REFERENCES

Manjunath G, Tighiouart H, Coresh J, et al.: Level of kidney function as a risk factor for cardiovascular outcomes in the elderly. Kidney Int 63:1121-1129, 2003a
U.S. Pat. No. 7,048,097 4. U.S. Pat. No. 7,048,907 B2 (Groman) Synthesis, compositions and methods for the measurement of the concentration of stable-isotope labeled compounds in life forms and life form excretory products.
Kobayashi et al: Detection of lymph node involvement in hematologic malignances using micromagnetic resonance lymphangiography with a gadolinium labelled dendrimer nanoparticles” Neoplasia. 2005 November, 7(11):984-91)
Choyke P L, Kobayashi H “Functional magnetic resonance imaging of the kidney using macromolecular contract agents” Abdom Imaging. 2006 March-April, 31(2): 224-31.
Manjunath G, Tighiouart H, Ibrahim H, et al.: Level of kidney function as a risk factor for atherosclerotic cardiovascular outcomes in the community. J Am Coll Cardiol 41:47-55, 2003b
Baran D, Tenstad O, Aukland K: Aprotinin uptake in the proximal tubules in the rat kidney. II. Uptake site relative to glomerulus. J Struct Biol 142:409-415, 2003a
Baran D, Tenstad O, Aukland K: Aprotinin uptake in the proximal tubules in the rat kidney I. Length of proximal tubular uptake segment. J Struct Biol 142:402-408, 2003b
Baran D, Tenstad O, Aukland K: Localization of tubular uptake segment of filtered Cystatin C and Aprotinin in the rat kidney. Acta Physiol (Oxf) 186:209-221, 2006
Roald A B, Aukland K, Tenstad O: Tubular absorption of filtered cystatin-C in the rat kidney. Exp Physiol 89:701-707, 2004a
Roald A B, Tenstad O, Aukland K: The effect of AVP-V2 receptor stimulation on local GFR in the rat kidney. Acta Physiol Scand 168:351-359, 2000
Roald A B, Tenstad O, Aukland K: The effect of AVP-V receptor stimulation on local GFR in the rat kidney. Acta Physiol Scand 182:197-204, 2004b
Tenstad O, Roald A B, Grubb A, et al.: Renal handling of radiolabelled human cystatin C in the rat. Scand J Clin Lab Invest 56:409-414, 1996
Tenstad O, Williamson H E, Aukland K: Repeatable measurement of local and zonal GFR in the rat kidney with aprotinin. Acta Physiol Scand 152:21-31, 1994a
Tenstad O, Williamson H E, Clausen G, et al.: Glomerular filtration and tubular absorption of the basic polypeptide aprotinin. Acta Physiol Scand 152:33-50, 1994b
Treeck B, Aukland K: Effect of L-NAME on glomerular filtration rate in deep and superficial layers of rat kidneys. Am J Physiol 272:F312-318, 1997
Treeck B, Roald A B, Tenstad O, et al.: Effect of exogenous and endogenous angiotensin II on intrarenal distribution of glomerular filtration rate in rats. J Physiol 541:1049-1057, 2002
Christiansen R E, Roald A B, Gjerstad C, et al.: Renal hemodynamics in young and old spontaneously hypertensive rats during intrarenal infusion of arginine vasopressin. Kidney Blood Press Res 24:176-184, 2001
Christiansen R E, Roald A B, Tenstad O, et al.: Renal hemodynamics during development of hypertension in young spontaneously hypertensive rats. Kidney Blood Press Res 25:322-328, 2002
Christiansen R E, Tenstad O, Leh S, et al.: Glomerular charge selectivity is impaired in hypertensive nephropathy. Nephrol Dial Transplant 19:1083-1091, 2004
Wang X, Aukland K, Bostad L, et al.: Autoregulation of total and zonal glomerular filtration rate in spontaneously hypertensive rats with mesangiolysis. Kidney Blood Press Res 20:11-17, 1997a
Wang X, Aukland K, Iversen B M: Autoregulation of total and zonal glomerular filtration rate in spontaneously hypertensive rats during antihypertensive therapy. J Cardiovasc Pharmacol 28:833-841, 1996
Wang X, Aukland K, Iversen B M: Acute effects of angiotensin II receptor antagonist on autoregulation of zonal glomerular filtration rate in renovascular hypertensive rats. Kidney Blood Press Res 20:225-232, 1997b
Wang X, Aukland K, Ofstad J, et al.: Autoregulation of zonal glomerular filtration rate and renal blood flow in spontaneously hypertensive rats. Am J Physiol 269:F515-521, 1995
AUKLAND, K., TENSTAD, O. & WIIG, H. (2001). Distribution spaces for hyaluronan andalbumin in rat tail tendons. Am J Physiol Heart Circ Physiol 281, H1589-1597.
BAKOUSH, O., TENCER, J., TORFFVIT, O., TENSTAD, O., SKOGVALL, I. & RIPPE, B. (2004). Increased glomerular albumin permeability in old spontaneously hypertensive rats. Nephrol Dial Transplant 19, 1724-1731.
BLETSA, A., BERGGREEN, E., FRISTAD, I., TENSTAD, O. & WIIG, H. (2006). Cytokine signalling in rat pulp interstitial fluid and transcapillary fluid exchange during lipopolysaccharide-induced acute inflammation. J Physiol 573, 225-236.
ERGA, K. S., PEEN, E., TENSTAD, O. & REED, R. K. (2000). Lactoferrin and antilactoferrin antibodies: effects of iron loading of lactoferrin on albumin extravasation in different tissues in rats. Acta Physiol Scand 170, 11-19.
GYENGE, C. C., TENSTAD, O. & WIIG, H. (2003). In vivo determination of steric and electrostatic exclusion of albumin in rat skin and skeletal muscle. J Physiol 552, 907-916.
KVEINE, M., TENSTAD, E., DOSEN, G., FUNDERUD, S. & RIAN, E. (2002). Characterization of the novel human transmembrane protein 9 (TMEM9) that localizes to lysosomes and late endosomes. Biochem Biophys Res Commun 297, 912-917.
LUND, U., RIPPE, A., VENTUROLI, D., TENSTAD, O., GRUBB, A. & RIPPE, B. (2003). Glomerular filtration rate dependence of sieving of albumin and some neutral proteins in rat kidneys. Am J Physiol Renal Physiol 284, F1226-1234.
NEGRINI, D., TENSTAD, O., PASSI, A. & WIIG, H. (2006). Differential degradation of matrix proteoglycans and edema development in rabbit lung. Am J Physiol Lung Cell Mol Physiol 290, L470-477.
NEGRINI, D., TENSTAD, O. & WIIG, H. (2003). Interstitial exclusion of albumin in rabbit lung during development of pulmonary oedema. J Physiol 548, 907-917.
NEGRINI, D., TENSTAD, O. & WIIG, H. (2003). Interstitial exclusion of albumin in rabbit lung measured with the continuous infusion method in combination with the wick technique. Microcirculation 10, 153-165.
ROSENGREN, B. I., RIPPE, B., TENSTAD, O. & WIIG, H. (2004). Acute peritoneal dialysis in rats results in a marked reduction of interstitial colloid osmotic pressure. J Am Soc Nephrol 15, 3111-3116.
TENSTAD, O., HEYERAAS, K. J., WIIG, H. & AUKLAND, K. (2001). Drainage of plasma proteins from the renal medullary interstitium in rats. J Physiol 536, 533-539.
TORLAKOVIC, E., TENSTAD, E., FUNDERUD, S. & RIAN, E. (2005). CD10+ stromal cells form B-lymphocyte maturation niches in the human bone marrow. J Pathol 205, 311-317.
WIIG, H., AUKLAND, K. & TENSTAD, O. (2003). Isolation of interstitial fluid from rat mammary tumors by a centrifugation method. Am J Physiol Heart Circ Physiol 284, H416-424.
WIIG, H., GYENGE, C. C. & TENSTAD, O. (2005). The interstitial distribution of macromolecules in rat tumours is influenced by the negatively charged matrix components. J Physiol 567, 557-567.
WIIG, H., KOLMANNSKOG, O., TENSTAD, O. & BERT, J. L. (2003). Effect of charge on interstitial distribution of albumin in rat dermis in vitro. J Physiol 550, 505-514.
WIIG, H., REED, R. K. & TENSTAD, O. (2000). Interstitial fluid pressure, composition of interstitium, and interstitial exclusion of albumin in hypothyroid rats. Am J Physiol Heart Circ Physiol 278, H1627-1639.
WIIG, H. & TENSTAD, O. (2001). Interstitial exclusion of positively and negatively charged IgG in rat skin and muscle. Am J Physiol Heart Circ Physiol 280, H1505-1512.
WIIG, H., TENSTAD, O. & BERT, J. L. (2005). Effect of hydration on interstitial distribution of charged albumin in rat dermis in vitro. J Physiol 569, 631-641.
Svarstad E, Hultstroffi, D., Jensen, D., Jenssen, G., Iversen, B. M. Renal artery thrombosis with acute failure after withdrawal of angiotensin converting enzyme inhibitor: a case report. Nephology Dialysis Transplantation 17:|−2, 2002.
Asarod, K, Iversen B. M, Hammerstrsm J, and Bostad, L and Jsrstad, S. Clinical outcome inpatients with Wegners graulomatosis treated with plasmapheresis Blood Purification 20:167-173.2002
Roald, A, Ofstad, J and Iversen B. M. Attenuated buffering of renal pressure variation in juxtamedullary cortex in the spontaneously hypertensive rat. Am. J. Physiol Renal Physiol 282: F506-F511. 2002.
Vikse, B E, Bostad, L, Aasarod, K, Dag Einar Lysebo, D E, and Iversen, B M. Prognostic factor in mesangioprqliferative glomerulonephritis. Nephrology Dialysis and transplantation 18: 17-23, 2002.
Svarstad E, Myking O, Ofstad J, and Iversen B. M. Effect of light excersise on renal hemodynamics in patients with hypertension and chronic renal disease. Scan J Urology Nephrology. 3 6: 6, 464-473, 2002
Christiansen, R F, Roald, A, Tenstad O and Iversen B M. Renal hemodynamics in young rats with genetic hypertension. Kidney and Blood Pressure Reserarch 2. 5: 322-328, 2002.
Vikse, B E, Aasarod K, Bostad L and Iversen B M. Prognostic factors in biopsy proven benign nephrosclerosis Nephrology Dialysis and Transplantation:2 003:18:517-23
Svarstad, E and Iversen B. M. Renal artery thombosis and acute renal failure after ACE inhibition. Nephrology Dialysis and Transplation 2004: 5 65.
Christiansen R. E, Tenstad O, Leh S and Iversen B M. Glomerular charges electivity impaired in hypertensive nephropathy. Nephrol Dialysis Transplantation: 19: I 083-1091, 2004
Vagnes O, Hansen F H, Christiansen R E, Gjerstad, C and Iversen B M: Age regulation of the VIa receptor in rats with genetic hypertension. Amer. J. Physiol 286:F997-F1003, 2004.
Svarstad, E, Iversen B M and Bostad, L. Bedsides tereomicroscopy of renal biopsies may lead to a rapid diagnosis of Fabry's disease. Nephrol. Dial. Transplant.; 1 9: 3202-3203, 2004.
Vikse B E, Vollset S E, Tell G S, Refsum H, Iversen B M. (2004). Distribution and determinants of serum creatinine in the general population: the Hordaland Health Study. Scand J Clin Lab Invest, 64, 1-14, 2004.
Ersver, E, Bertelsen, L-T, Espenes, L. CB, redholt, T, BAe, SO, Iversen, B M, Bruserud, O, Ulvestad, E, Gjerts-enB, T. Characterization of ribosomal P autoantibodies in relation to cell destruction and autoimmune diseaqe. Scand. Jour. Immunology 60,189-198, 2004.
Strommen, K., Stormark, T A., Iversen, B M. and Matre K. Volume estimation of small phantoms and rat kidneys using three-dimensional ultrasonography and position sensor. 30(9), 2004
Svarstad E, Iversen B M, Bostad L. Bedsides tereomicroscopy of renal biopsies may lead to a rapid diagnosis of Fabry's disease. Nephrol Dial Transplant. 2004 December; 19(12):3202-3.
Hansen F H, Vagnes O B, Iversen B M. Enhanced response to arginine vasopressin (AVP) in the interlobular artery (ILA) from the spontaneously hypertensive rat (SHR). Am J Physiol Renal Physiol 288 (6):F]oa9-56, 2005.
Bivol L M, Vagnes O B, Iversen B M. The renal vascular response to ANG II injection is reduced in the non-clipped kidney of two-kidney, one clip hypertension. Am J Physiol Renal Physiol. Am J Physiol Renal Physiol. A; 289(2):F393-400, 2005
Vagnes O, Hansen F H, Feng J J, Iversen B M, Arendshorst W J. Enhance d Ca2+ response to AVP in preglomerular vessels from rats with genetic hypertension during different hydration States Am J Physiol R enal Physiol. 88(5):F1023-31, 2005.
Svarstad, E., BostadL., Kaabae, A, Houge, G., Tsndel, C., Lyngdal, P. T. and Iversen, B. M: Focal and segmental glomerular sclerosis (FSGS) in a man and a woman with Fabry's Disease Clinical nephrology 63:394-400, 2005
Svarstad, E, Urheim L and B. M. Iversen:Critical renal artery stenosis may cause a spectrum of cardiorenal failure and associated thromboembolic events. Clinical Nephrology 63, 487-492, 2005.
Leh S, Vaagnes O, Margolin S B, Iversen B M, Forslund T. Pirfenidone and candesartanameliorate morphological damage in mild chronic anti-GBM nephritis in rats. Nephrol Dial Transplant 20(1)7 I-82, 2005.
Ofstad J, Iversen B M. Glomerular and tubular damage in normotensive and hypertensive rats. Am J Physiol Renal Physiol.; 288(a):F665-7 2, 2005.
Svarstad, E., Bostad L., Kaabae, 6, Hougq G., Tondel, C., Lyngdal, P. T. and Iversen, B. M: Focal and segmental glomerular sclerosis (FSGS) in a man and a woman with Fabry's Disease Clinical Nephrology 63: 394-401, 2005
Vikse, B. E., Irgens, L., Bostad, L. and Iversen B. M. Adverse perinatal outcome and late kidney disease in the mother. J. Amer. Soc Nephrology. 2006
Helle, F, Vagnes O and Iversen B M: Angiotensin II indiced calcium-signalling in-two kidney-one clip hypertension Amer J. Physiolol, Renal, 2006
Erik Ilsø Christensen & Henrik Birn, Nature Reviews Molecular Cell Biology 3, 258-268, April 2002

### EXAMPLES

### Example 1

### Gd-DTPA-Aprotinin Protocol

### Materials:

DTPA (Diethylenetriaminepentaacetic acid dianhydride, Sigma D-6148, C₁₄H₁₉N₃O₈, molecular weight 357.32).
aprotinin from bovine lung, Sigma A4529 lyophilized powder, 3-7 TIU/mg solid, molecular weight 6511.44).
HEPES (Sigma, H7523, 4-(2-Hydroxyethyl)piperazine-1-ethanesulfonic acid, C₈H₁₈N₂O₄S, molecular weight 238.30).
Citric acid (Sigma C0759, HOC(COOH)(CH₂COOH)₂ molecular weight 192.12).
Citrate tribasic dehydrate (Sigma S4641, HOC(COONa)(CH₂COONa)₂.2H₂O, molecular weight 294.10).
DMSO (Sigma Dimethyl sulfoxide, D8418, (CH₃)₂SO, molecular weight 78.13).
Dialysis Membrane (Spectra/Por 3 Tubing: 3.5 k MWCO Regenerated Cellulose, Cat. No.: 132720).
NTA, (Sigma, N0128, Nitrilotriacetic acid disodium salt, C₆H₇NO₆Na₂, molecular weight 235.10
GdCl₃ (MP Biomedicals Cat. No.: 203712, gadolinium chloride, Cl₃GdH₁₂O₆, molecular weight: 371.7).
Gadolinium-153 Radionuclide in 0.5N HCl (PerkinElmer NEZ142001MC)

### Day 1: Production of DTPA-aprotinin.

Dissolve 11 mg aprotinin in 250 μl 0.1 M HEPES buffer at pH 8.8.

Dissolve 0.48 g DTPA in 1 ml DMSO.

Add 25 μl of the DMSO/DTPA Solution to the Aprotin solution and dialyse overnight against 1000 ml 0.1M citrate buffer adjusted to pH 6.5 by using 1% Citric acid.

### Day 2: Second Dialysis

Change to fresh citrate buffer.

### Day 3: Third Dialysis

Change to fresh citrate buffer.

### Day 4: Production of Gd-DTPA-aprotinin.

Make Gd(NTA)₂ by dissolving 260 mg GdCl₃ in 4 ml pH 1.0 HCl. Ad 100 μCi Gadolinium-153 if biological screening using gamma detection is desired.

Add 494 mg NTA (Get white precipitate). Add 3M NaOH dropwise until pH is 4.9.

Add 20 μl Gd(NTA)₂ to the dialysed DTPA-aprotinin solution with stirring.

Continue stirring in for 3 h and dialyse overnight against 1000 ml 0.1M citrate buffer as described above.

### Day 5: Second Dialysis

Change to fresh citrate buffer.

### Day 6: Third Dialysis

Change to fresh citrate buffer.

Gd-DTPA-aprotinin is stable in citrate buffer at 4-8° C. for at least 3 months.

### Example 2

### Gd-DTPA-Lysozyme Protocol

### Materials:

DTPA (Diethylenetriaminepentaacetic acid dianhydride, Sigma D-6148, C₁₄H₁₉N₃O₈, molecular weight 357.32).
Lysozyme from chicken egg white, Sigma L6876, lyophilized powder, molecular weight 14.7 kDa.
HEPES (Sigma, H7523, 4-(2-Hydroxyethyl)piperazine-1-ethanesulfonic acid, C8H18N2O4S, molecular weight 238.30).
Citric acid (Sigma C0759, HOC(COOH)(CH2COOH)2 molecular weight 192.12).
Citrate tribasic dehydrate (Sigma S4641, HOC(COONa)(CH2COONa)2.2H2O, molecular weight 294.10).
DMSO (Sigma Dimethyl sulfoxide, D8418, (CH3)2SO, molecular weight 78.13).
Dialysis Membrane (Spectra/Por 3 Tubing: 3.5 k MWCO Regenerated Cellulose, Cat. No.: 132720).
NTA, (Sigma, N0128, Nitrilotriacetic acid disodium salt, C6H7NO6Na2, molecular weight 235.10
GdCl3 (MP Biomedicals Cat. No.: 203712, gadolinium chloride, Cl3GdH12O6, molecular weight: 371.7).
Gadolinium-153 Radionuclide in 0.5N HCl (PerkinElmer NEZ142001MC)

### Day 1: Production of DTPA-Lysozyme

Dissolve 220 mg Lysozyme in 5 ml 0.1 M HEPES buffer at pH 8.8.

Dissolve 0.48 g DTPA in 1 ml DMSO.

Add 100 μl of the DMSO/DTPA solution to the Lysozyme solution and adjust pH to 8.8. Repeat this step five times so that totally 500 μl DSMO/DTPA is added. Dialyse overnight against 1000 ml 0.1M citrate buffer adjusted to pH 6.5 by using 1% Citric acid.

### Day 2: Second Dialysis

Change to fresh citrate buffer.

### Day 3: Third Dialysis

Change to fresh citrate buffer.

### Day 4: Production of Gd-DTPA-Lysozyme

Make Gd(NTA)2 by dissolving 260 mg GdCl3 in 4 ml pH 1.0 HCl. Ad 100 μCi Gadolinium-153 if biological screening using gamma detection is desired.

Add 494 mg NTA (get white precipitate). Add 3M NaOH dropwise until pH is 4.9.

Add 400 μl Gd(NTA)2 to the dialysed DTPA-Lysozyme solution with stirring. Continue stirring in for 3 h and dialyse overnight against 1000 ml 0.1M citrate buffer as described above.

### Day 5: Second Dialysis

Change to fresh citrate buffer.

### Day 6: Third Dialysis

Change to fresh citrate buffer.

Gd-DTPA-Lysozyme is stable in citrate buffer at 4-8° C. for at least 3 months.

### Example 3

### Study of Glomerular Filtration Rate in a Patient

In one example, provided in order to illustrate and not to limit, a complex comprising aprotinin linked to Gadolinium is administered intravenously to a patient and MR imaging performed. Imaging visualises the glomerular filtration rate of said patient. Subsequently, a complex comprising lysozyme linked to Gadolinium is administered intravenously to the same patient and MR imaging performed. The images acquired in the two scans are compared. Deficient uptake of complex comprising a lysozyme derivative (tubular injury inhibits absorption of this complex) in the absence of deficiency of the complex comprising aprotinin (absorbed even by injured tubules) indicates damage (e.g. after acute ischemia) locally to tubuli which has not yet resulted in lowered glomerular filtration rate.

### Example 4

### Study of Alterations of Intrarenal Distribution Og GFR in a Patient

In another illustrative example aprotinin linked to gadolinium is administered intravenously to a patient suffering from unilateral renal artery stenosis in order to measure the intracortical distribution of glomerular filtration rate in the two kidneys. Unilateral renal artery stenosis is known to result in early perturbations of the nephrons in outer cortical layers of the stenotic kidney. The inner cortical nephrons of the contra lateral kidney exposed to hypertension are on the other hand the first to develop glomerulosclerosis. Thus, the inner cortex to outer cortex ratio of local glomerular filtration rate in the two kidneys, being similar in healthy subjects and altered in opposite directions in unilateral renal artery stenosis, would allows very early detection of functional abnormality and help the clinicians to decide when surgical intervention is favourable.

1-64. (canceled)

65. A complex suitable for use as a contrast agent which comprises:
a biomolecule; and
a marker associated with said biomolecule.

66. The complex according to claim 65 wherein the biomolecule associated with the marker forms supracomplexes.

67. The complex according to claim 65, wherein the marker is selected from the group consisting of a paramagnetic marker and a radioactive marker.

68. The complex according to claim 67 wherein the marker is a paramagnetic marker and the magnetic moment of the paramagnetic marker is in the range of from 6 to 9

69. The complex according to claim 67 wherein the marker is a paramagnetic marker and the magnetic moment of said paramagnetic substance is greater than 7.

70. The complex according to claim 67 wherein the marker is a paramagnetic marker selected from the group consisting of aluminium, barium, calcium, dysprosium, gadolinium, magnesium, manganese, oxygen, platinum, sodium, strontium, technetium and uranium.

71. The complex according to claim 65 wherein, the marker comprises gadohnium.

72. The complex according to claim 65 wherein the biomolecule is selected from the group consisting of modified and unmodified nucleic acids, polypeptides, polysaccharides, and lipids.

73. The complex according to claim 65 wherein the biomolecule is a polypeptide.

74. The complex according to claim 73 wherein the polypeptide is a globular polypeptide.

75. The complex according to claim 65 wherein the complex is suitable for imaging a subject's kidney.

76. The complex according to claim 65 wherein the complex accumulates in a subject's renal cortex.

77. The complex according to claim 65 wherein the biomolecule hinds to Megalin and/or Cubulin.

78. The complex according to claim 65 wherein the biomolecule has a molecular weight ranging from 1 kDa to 50 kDa

79. The complex according to claim 78 wherein the biomolecule as a molecular weight ranging from 5 kDa to 40 kDa.

80. The complex according to claim 65 wherein the biomolecule has an isoelectric point ranging from 7.5 to 11.5.

81. The complex according to claim 80 wherein the biomolecule has an isoelectic point ranging from 8 to 10.5.

82. The complex according to claim 65 wherein the biomolecule is selected from the group consisting of i) aprotinin, or a fragment or a derivative thereof, ii) chyrmotryspsinogen A, or a fragment or a derivative thereof, iii) lysozyme, or a fragment or a derivative thereof, iv) ovalbumin, or a fragment or a derivative thereof, v) ribonuclease, or a fragment or a derivative thereof, vi) cytochrome c, or a fragment or a derivative thereof, vii) cystatin c, or a fragment or a derivative thereof, and viii) angiotensin, or a fragment or a derivative thereof.

83. A composition for use as a contrast agent comprising:
one or more complexes, which may be the same or different, each of which comprise a biomolecule, a marker, wherein the biomolecule is associated with the marker; and
a carrier.

84. The composition according to claim 83 wherein the composition comprises at least two different complexes.

85. The composition of claim 84 where the at least two different complexes comprise different biomolecules.

86. The composition of claim 84 wherein the at least two different complexes comprise different paramagnetic markers.

87. The composition according to claim 83 wherein said biomolecule of said one or more complexes is selected from the group consisting of aprotinin and lysozyme.

88. The composition according to claim 83 wherein one or more of said one or more complexes comprise at least one of aprotinin as said biomolecule, and gadolinium as said marker.

89. The composition according to claim 83 wherein at least one of said one or more complexes comprises gadolinium as said marker and lysozyme as said biomolecule.

90. A method for imaging of a tissue, organ or compartment thereof, comprising the steps of
a. visualising one or more complexes each of which comprises a biomolecule and a marker after the one or more complexes have been administered to a subject and have become associated with a tissue, organ or compartment thereof, and
b. capturing images of said tissue, organ or compartment thereof while said one or more complexes are associated with said tissue, organ, or compartment thereof.

91. The method of imaging set forth in claim 90 where at least one of said one or more complexes includes gadolinium as said marker.

92. The method of imaging set forth in claim 90 wherein at least one of said one or more complexes includes aprotinin or lysozyme as said biomarker.

93. The method of imaging set forth in claim 90 wherein said step of capturing images is performed using one of magnetic resonance imaging (MRI) and radiograph based imaging.

94. The method of imaging set forth in claim 90 wherein said tissue, organ, or compartment thereof is selected from the group consisting of kidney, small intestine and compartments thereof.

95. The method of imaging set forth in claim 90 wherein said visualizing step includes visualizing at least two different complexes which have been administered to said subject simultaneously or sequentially in any order.

96. The method of imaging set forth in claim 95 wherein said at least two different complexes include a first complex which includes gadolinium and aprotin and a second complex which includes gadolinium and lysozyme.

97. The method of imaging set forth in claim 95 wherein said steps of visualizing and imaging and capturing images are repeated in sequence with said first complex and said second complex.

98. The method of claim 97 wherein both times capturing images is performed in sequence magnetic resonance imaging (MRI) is used.

99. A method of determining total or glomular filtration rate of a subject's kidney or one or more volumes thereof, comprising the steps of
providing a subject at one or more specified time intervals with specified amounts of a one or more complexes each of which
i) comprises a biomolecule and a marker,
ii) is freely filtered in the glomeruli, and
iii) is reabsorbed and retained in tubular cells;
capturing images of the subject's kidney or one more volumes thereof; and
based on the images captured in the capturing step and on the specified tune intervals and specified amounts in said providing step, computing one or more of a total and regional glomular filtration rate as a ratio of an amount of a marker provided per kidney volume of interest and time integrated plasma concentration of the marker from the time of providing to the time of capturing an image.

100. The method of claim 99 wherein one of said one or more complexes is less than 20 kDA.

101. The method of claim 99 wherein one of said one or more complexes includes gadolinium as said marker.

102. A method for identifying and/or evaluating proteinurea in a subject, comprising the steps of:
providing a subject with a one or more complexes each of which
i) comprises a biomolecule and a marker,
ii) is 20 KDa to 100 KDa in size;
capturing images of the subject's kidney or portions thereof; and
determining, based on said images, whether said one or more complexes has leaked through damaged glomerular capillary walls and accumulated in tubular cells.

103. The method of claim 102 wherein said determining step includes quantifying an amount of said marker detected in said determining step and comparing it to a provided amount of said one or more complexes in said providing step.

104. The method of claim 102 wherein one of said one or more complexes includes gadolinium as said marker.
